SEQUENCE LISTING (10 sequences)

SEQ ID NO: 1 - DNA, 1997 bp, Agrobacterium tumefaciens
gatcctgttacaagtattgcacgttttataaattgcatattaatgcaatcttgatgtttaacaacgaaggtaatggcgtaaaagaaaaaatgtatgttattgtattgatctttcattatgttgaagtgtgccataatatgatgtataagtaaaatatcaactgtcgcatttattgaaatggcactgttatttcaaccatatctttgattctgtgacaatgacaacgactgcaagaagtaaataatagacgccgttgttaaaggattgctatcatatgtgcctaactatagggacatttacgtcaattgtgaaatagtcgcccttattttgacgtctcacctaatcaaatattacaaacgatctcactctgtcgccagcaatggtgtaatcagcgcagacaagtggcagtaaagcgcggaaaaacgtccccgagtggcatgaatagctgcctctgtattgctgatttagtcagccttatttgacttaagggtgccctcgttagtgacaaattgctttcaaggagacagccatgccccacactgtgttgaaaaacaaattgccctttggggagacggtaaagccagttgctcttcaataaggaatctcgaggaggcaatataaccgcctctggtagtacacttctctaatccaaaaatcaatttgtattcaagataccgcaaaaaacttatggatctgcgtctaattttcggtccaacttgcacaggaaagacgtcgaccgcggtagctcttgcccagcagactgggcttccagtcctttcgctcgatcgggtccaatgttgtcctcagctgtcaaccggaagcggacgaccaacagtggaagaactgaaaggaacgagccgtctataccttgatgatcggcctctggtgaagggtatcatcgcagccaagcaagctcatgaaaggctgatgggggaggtgtataattatgaggcccacggcgggcttattcttgagggaggatctatctcgttgctcaagtgcatggcgcaaagcagttattggagtgcggattttcgttggcatattattcgccacgagttagcacacgaggagaccttcatgaacgtggccaaggccagagttaagcagatgttacgccccgcttcaggcctttctattatccaagagttggttgatctttggaaagagcctcggctgaggcgcatactgaaagagatcgatggatatcgatatgccatgttgtttgttagccagaaccagatcacatccgatatgctattgcagcttgacgcagatatggaggataagttgattcatgggatcgctcaggagtatctcatccatgcacgccgacaagaacagaaattccctcgagttaacgcagccgcttacgacggattcgaaggtcatccattcggaatgtattagtttgcaccagctccgcgtcacacctgtcttcatttgaataagatgttcgcaattgtttttagctttgtcttgttgtggcagggcggcaagtgcttcagacatcatcctgttttcaaaatttatgctggtgaacagcttcttaattcctttggaaatactagactgcgtcttaaaattacgatgtctagatgaagatgtgattgtaaaataacctatttaagttttcatttagaacataagttttatgaatgttcttccattttcgtcatcgaacgaataagagtaaatacaccttttttaacattataaaaataagttcttatacgttgtttatacaccgggaatcatttccattattttcgcgcaaaaagtcacggatattcgtgaaagcgacaaaaactgcgaaatttgcggggagtgtcttcagtttgcctattaatatttagtttgacactaattgttaccattgcagccaagctcagctgtttcttttcttaaaaacgcaggatcgaaagagcatgactcggcaaggttggcttgtaccatgcatttctcatgccaaagatgatcaactgcaggtcgactct

SEQ ID NO: 2 - protein, 240 aa, Agrobacterium tumefaciens
MDLRLIFGPTCTGKTSTAVALAQQTGLPVLSLDRVQCCPQLSTGSGRPTVEELKGTSRLYLDDRPLVKGIIAAKQAHERLMGEVYNYEAHGGLILEGGSISLLKCMAQSSYWSADFRWHIIRHELAHEETFMNVAKARVKQMLRPASGLSIIQELVDLWKEPRLRRILKEIDGYRYAMLFVSQNQITSDMLLQLDADMEDKLIHGIAQEYLIHARRQEQKFPRVNAAAYDGFEGHPFGMY

SEQ ID NO: 3 - DNA, 2268 bp, Agrobacterium tumefaciens
atgtcagcttcacctctccttgataaccagtgcgatcatttctctaccaaaatggtggatctgataatggtcgataaggctgatgaattggaccgcagggtttccgatgccttctcagaacgtgaagcttctaggggaaggaggattactcaaatctccggcgagtgcagcgctgggttagcttgcaaaaggctggccgacggtcgctttcccgagatctcaactggtgagaaggtagcagccctctccgcttacatctatgttggcaaggaaattctggggcggatacttgaatcggaaccttgggcgcgagcaagagtgagtggtctcgttgccatcgaccttgcaccattttgtatggatttctccgaagcacaacttctccaaaccctgtttttgctgagcggtaaaagatgtgcatccagcgatcttagtcatttcgtggccatttcaatctctaagactgcccgctcccgaaccctgcaaatgccgccgtacgagaaaggcacgacgaaacgcgttaccgggtttaccctgacccttgaagaggccgtaccatttgacatggtagcttatggtcgaaacctgatgctgaaggcttcggcaggttcctttccaacaattgacttgctctatgactacagatcgttttttgaccaatgttccgatattggacggatcggcttctttccggaagatgttcctaagccgaaagtggcgatcattggcgctggcatttccggactcgtggtagcaagcgaactgcttcatgctggtgtagacgatgttacaatatatgaagcaagtgatcgggttggaggcaagctttggtcacatgctttcaaggatgctcccagcgtggtggccgaaatgggggcgatgcgatttcctcctgctgcatcgtgcttgtttttcttcctcgagcggtacggcctgtcttcgatgaggccgttcccaaatcccggcacagtcgacactaacttggtctaccaaggcctccgatacgtgtggaaagccgggcagcagccaccgaagctgttccatcgcgtttacagcggttggcgtgcgttcttgagggacggtttccatgagggagatattgtgttggcttcgcctgttgttattactcaagccttgaaatcaggagacattaggcgggctcatgactcctggcaaacttggctgaaccgtttcgggagggagtccttctcttcagcgatagagaggatctttctgggcacgcatcctcctggtggtgaaacatggagtttccctcatgattgggacctattcaagctaatgggaataggatctggcgggtttggtccagtttttgaaagcgggtttattgagatccttcgcttggtcataaacggatatgaagaaaatcagcggatgtgctctgaaggaatctcagaacttccacgtcgaatagcctctcaagtggttaacggtgtgtctgtaagccagcgtatacgccatgttcaagtcagggcgattgagaaggaaaagacaaaaataaagataaggcttaagagcgggatatctgaactttatgataaggtggtggttacatctggactcgcaaatatccaactcaggcattgtctgacatgcgataccaccatttttcgtgcaccagtgaaccaagcggttgataacagccatatgacaggctcgtcaaaactctttctgctgactgaacgaaaattttggttagaccatatcctcccgtcctgtgtcctcatggacgggatcgcaaaagcagtgtactgcttggactatgagccgcaggatccgaatggtaaaggtctggtgccccccacttatacatgggaggacgactcccacaagctgttggcggttcccgacaaaaaagagcgattctgtctgctgcgggacgcaatttcgagatctttcccggcgtttgcccagcatctagttcctgcctgcgctgattacgaccaaaatgttgttcaacatgattggcttacagacgagaatgccgggggagctttcaaactcaaccggcgtggcgaggatttttattctgaagaacttttctttcaagcgctggacatgcctaatgataccggagtttacttggcgggttgcagttgttccttcaccggtggatgggtggagggcgctattcagaccgcgtgtaacgccgtctgtgcaattatccacaattgtggaggtattttggcaaaggacaatcctctcgaacactcttggaagagatataactaccgcaatagaaattaa

SEQ ID NO: 4 - protein, 755 aa, Agrobacterium tumefaciens
MSASPLLDNQCDHFSTKMVDLIMVDKADELDRRVSDAFSEREASRGRRITQISGECSAGLACKRLADGRFPEISTGEKVAALSAYIYVGKEILGRILESEPWARARVSGLVAIDLAPFCMDFSEAQLLQTLFLLSGKRCASSDLSHFVAISISKTARSRTLQMPPYEKGTTKRVTGFTLTLEEAVPFDMVAYGRNLMLKASAGSFPTIDLLYDYRSFFDQCSDIGRIGFFPEDVPKPKVAIIGAGISGLVVASELLHAGVDDVTIYEASDRVGGKLWSHAFKDAPSVVAEMGAMRFPPAASCLFFFLERYGLSSMRPFPNPGTVDTNLVYQGLRYVWKAGQQPPKLFHRVYSGWRAFLRDGFHEGDIVLASPVVITQALKSGDIRRAHDSWQTWLNRFGRESFSSAIERIFLGTHPPGGETWSFPHDWDLFKLMGIGSGGFGPVFESGFIEILRLVINGYEENQRMCSEGISELPRRIASQVVNGVSVSQRIRHVQVRAIEKEKTKIKIRLKSGISELYDKVVVTSGLANIQLRHCLTCDTTIFRAPVNQAVDNSHMTGSSKLFLLTERKFWLDHILPSCVLMDGIAKAVYCLDYEPQDPNGKGLVPPTYTWEDDSHKLLAVPDKKERFCLLRDAISRSFPAFAQHLVPACADYDQNVVQHDWLTDENAGGAFKLNRRGEDFYSEELFFQALDMPNDTGVYLAGCSCSFTGGWVEGAIQTACNAVCAIIHNCGGILAKDNPLEHSWKRYNYRNRN

SEQ ID NO: 5 - DNA, 1404 bp, Agrobacterium tumefaciens
ctaattaaaagcatcgggaaaggaagaaaaatttatagctttttctaatgctgccccgattgctaacagacggtggtctgaccccgctaatccatcaatttccattccaacaggcaagcgatcaggtgtaaggcaggcaggaaggctcaacccaggtaggcctgcgttgctgcttgggtccacatttcgcacgtagatcttgaaagtgttcatcattgagccattgtggatgactgacgactcctgacctatggctttggccgctaagggtgcagttgggaaaaggattgcatctaactgatagagtctgaagtaattccgataagtggcctggagccttggcctgaaggattgacgcgccagttcatattcatcgttggaaatttgatgcccatcaatttgcgcactgacaatgttcgctacatcggggctacgaattcctttgataacgtcagaaaaagaaactgttcccacaaaatcgtcgagatacttttttagagcgtgtggaaattcgtaaagcgcaattggcaaacttgccccactgttcaattcctctaggtgggggatgtcggcttcaacaaaggttacgcctctgttggctagcaagcgaatcgtcgtttcagctgcgaaggccacatcagcatcaaggtcatcgtaaaagtaggtagtggggaggccgatccgaagccccttcagcggcatgggtgaaattttcgccgaccgtccggaaatcacctggtcgaggattataacatcggctacgcactgcgctatgattccggcggtgtcccgggtggggctgaacggtattatccgatctcttggatatcgaccaagcgtcggtcgaaatcctactacgccacacagggctgccggtaggcgaacagatgcaccggtatccgtgcctatgccgcctaacatcaatcggcttgccaccgcagcagccacaccaccgcttgaaccccctggtatcagacttggattccacgggttccgcaccgcaccggtggcatagttgttgctcgtaattccaaacgataactcatgcatgtttcccgaggcacccggcagtgctccagctgaaaaaagtctttctgcgacgcgggatggtatctttggcaagtggtttatcagcgccggagtagcagcgcttgtaggaaatacgccggtcgcgatgttcgccttaaaacagagtggaatgccgcaaagacctaatccggcgtttccatgacgatcattttttttggcgcttcgccgcaagccatcccagtctgtagccagaagggcatttaatggttttgcagcttggcaacgcgctatcagagtttctactagttctaagcaggagtagtctttccgtctcaggcgttctagggtttgtgctaacgaggtaatgggcaccat

SEQ ID NO: 6 - protein, 467 aa, Agrobacterium tumefaciens
MVPITSLAQTLERLRRKDYSCLELVETLIARCQAAKPLNALLATDWDGLRRSAKKNDRHGNAGLGLCGIPLCFKANIATGVFPTSAATPALINHLPKIPSRVAERLFSAGALPGASGNMHELSFGITSNNYATGAVRNPWNPSLIPGGSSGGVAAAVASRLMLGGIGTDTGASVRLPAALCGVVGFRPTLGRYPRDRIIPFSPTRDTAGIIAQCVADVIILDQVISGRSAKISPMPLKGLRIGLPTTYFYDDLDADVAFAAETTIRLLANRGVTFVEADIPHLEELNSGASLPIALYEFPHALKKYLDDFVGTVSFSDVIKGIRSPDVANIVSAQIDGHQISNDEYELARQSFRPRLQATYRNYFRLYQLDAILFPTAPLAAKAIGQESSVIHNGSMMNTFKIYVRNVDPSSNAGLPGLSLPACLTPDRLPVGMEIDGLAGSDHRLLAIGAALEKAINFSSFPDAFN

SEQ ID NO: 7 - DNA, 2095 bp, Agrobacterium tumefaciens
gccagaaaagattggcctttgtgtagataactaacgacatcaaccgtttctatttaagttgagaatcggggaacatactgttcattgacatcccaatggaaagcgacaatttgggcattttttaatacaatattcacgcaggtccacctcgccgctcaatcaagttgaatattgattgatacaatactgtttccgttcgaaaaagcgaccttagtgcctccaataattagtatcatacgccgtgattgcggaattccctccaataatcgcgcaatttgggtccaagcgcggtatatttatttaatctgaaatggccccgaaatccccaaaccagaaaaatggtgcccattacctcgttagcacaaaccctagaacgcctgagacggaaagactactcctgcttagaactagtagaaactctgatagcgcgttgccaagctgcaaaaccattaaatgcccttctggctacagactgggatggcttgcggcgaagcgccaaaaagaatgatcgtcatggaaacgccggattaggtctttgcggcattccactctgttttaaggcgaacatcgcgaccggcgtatttcctacaagcgctgctactccggcgctgataaaccacttgccaaagataccatcccgcgtcgcagaaagacttttttcagctggagcactgccgggtgcctcgggaaacatgcatgagttatcgtttggaattacgagcaacaactatgccaccggtgcggtgcggaacccgtggaatccaagtctgataccaggaggctcaagcggtggtgtggctgctgcggtggcaagccgattgatgttaggcggcataggcaccgataccggtgcatctgttcgcctacccgcagccctgtgtggcgtagtaggatttcgaccgacgcttggtcgatatccaagagatcggataataccggtcagccccacccgggacaccgccggaatcatagcgcagtgcgtagccgatgttataatcctcgaccaggtgatttctggacggtcggcgaaaatttcacccatgccgctggaggggcttcggatcggcctccccactacctacttttacgatgaccttgatgctgatgtggccttcgcagctgaaacgacgattcgcttgctagccaacagaggcgtaacctttgttgaagccgacatcccccacctagaggaattgaacagtggggcaagtttgccaattgcgctttacgaatttccacacgctctaaaaaagtatctcgacgattttgtgggaacagtttctttttctgacgttatcaaaggaattcgtagccccgatgtagcgaacattgtcagtgcgcaaattgatgggcatcaaatttccaacgatgaatatgaactggcgcgtcaatccttcaggccaaggctccaggccacttatcggaattacttcagactctatcagttagatgcaatccttttcccaactgcacccttagcggccaaagccataggtcaggagtcgtcagtcatccacaatggctcaatgatgaacactttcaagatctacgtgcgaaatgtggacccaagcagcaacgcaggcctacctgggttgagccttcctgcctgccttacacctgatcgcttgcctgttggaatggaaattgatggattagcggggtcagaccaccgtctgttagcaatcggggcagcattagaaaaagctataaatttttcttcctttcccgatgcttttaattagctgtatgccacaccacttattgtctaataaatctgcacttcagttagcattatctgaatgtgatagaaatttcttcagacaggcttgtcgttcccgaaatcaaaacagctacttgatatatatattctacagttattgcgtctccaaggaatagaatacgcattatcttttgactcaatagtgaattagattcccaaataaaattaaagtatagaatttcgtaggctgcacctacaatttgttttgagcgccgaacggctcaaagattagacaggggccccaagccaaaaatattaaccatatatatctttagcaaataatttatcactcataaggcacgcaacaagacaacg

SEQ ID NO: 8 - DNA, 1404 bp, Agrobacterium tumefaciens
atggtgcccattacctcgttagcacaaaccctagaacgcctgagacggaaagactactcctgcttagaactagtagaaactctgatagcgcgttgccaagctgcaaaaccattaaatgcccttctggctacagactgggatggcttgcggcgaagcgccaaaaagaatgatcgtcatggaaacgccggattaggtctttgcggcattccactctgttttaaggcgaacatcgcgaccggcgtatttcctacaagcgctgctactccggcgctgataaaccacttgccaaagataccatcccgcgtcgcagaaagacttttttcagctggagcactgccgggtgcctcgggaaacatgcatgagttatcgtttggaattacgagcaacaactatgccaccggtgcggtgcggaacccgtggaatccaagtctgataccaggaggctcaagcggtggtgtggctgctgcggtggcaagccgattgatgttaggcggcataggcaccgataccggtgcatctgttcgcctacccgcagccctgtgtggcgtagtaggatttcgaccgacgcttggtcgatatccaagagatcggataataccggtcagccccacccgggacaccgccggaatcatagcgcagtgcgtagccgatgttataatcctcgaccaggtgatttctggacggtcggcgaaaatttcacccatgccgctggaggggcttcggatcggcctccccactacctacttttacgatgaccttgatgctgatgtggccttcgcagctgaaacgacgattcgcttgctagccaacagaggcgtaacctttgttgaagccgacatcccccacctagaggaattgaacagtggggcaagtttgccaattgcgctttacgaatttccacacgctctaaaaaagtatctcgacgattttgtgggaacagtttctttttctgacgttatcaaaggaattcgtagccccgatgtagcgaacattgtcagtgcgcaaattgatgggcatcaaatttccaacgatgaatatgaactggcgcgtcaatccttcaggccaaggctccaggccacttatcggaattacttcagactctatcagttagatgcaatccttttcccaactgcacccttagcggccaaagccataggtcaggagtcgtcagtcatccacaatggctcaatgatgaacactttcaagatctacgtgcgaaatgtggacccaagcagcaacgcaggcctacctgggttgagccttcctgcctgccttacacctgatcgcttgcctgttggaatggaaattgatggattagcggggtcagaccaccgtctgttagcaatcggggcagcattagaaaaagctataaatttttcttcctttcccgatgcttttaattag

SEQ ID NO: 9 - protein, 467 aa, Agrobacterium tumefaciens
MVPITSLAQTLERLRRKDYSCLELVETLIARCQAAKPLNALLATDWDGLRRSAKKNDRHGNAGLGLCGIPLCFKANIATGVFPTSAATPALINHLPKIPSRVAERLFSAGALPGASGNMHELSFGITSNNYATGAVRNPWNPSLIPGGSSGGVAAAVASRLMLGGIGTDTGASVRLPAALCGVVGFRPTLGRYPRDRIIPVSPTRDTAGIIAQCVADVIILDQVISGRSAKISPMPLEGLRIGLPTTYFYDDLDADVAFAAETTIRLLANRGVTFVEADIPHLEELNSGASLPIALYEFPHALKKYLDDFVGTVSFSDVIKGIRSPDVANIVSAQIDGHQISNDEYELARQSFRPRLQATYRNYFRLYQLDAILFPTAPLAAKAIGQESSVIHNGSMMNTFKIYVRNVDPSSNAGLPGLSLPACLTPDRLPVGMEIDGLAGSDHRLLAIGAALEKAINFSSFPDAFN

SEQ ID NO: 10 - DNA, 21126 bp, Agrobacterium rhizogenes
ggccgcaggatttcgttcgtcgtgcgtgatgagatcgataaatgtttatcgacgaggacaagatcgacgatgcggttcttgcgctgttgtagtgacgctccacaacgagtgttgcgccgtgaaaggctttgactgggccgcgacggaccgcctttgcaggaagggttcggtcggcgatcccgtcaataaatcgaagctattgatcctgacggataaaggtctgcgtcgatcggaggagctattccgacagctgtttacgcgctagccattggccgacggtctttgcgccctccattcccacggcgtagttaatgccggcggggacgggagtgtctactatgtgcaagcacgtcggcgaaccatgccttcggattaatgtcgttcagacgggcggtcgtaagttgaatgagtatgactgccgcatggtcagcgccgcgttgggagccggcagatgtccagtcgcggcgcctcaaggccatcacatgttcactctgtggccagaaggcgtcgctccttgggtggcaggatatattgtgatgtaaacagattagatatggacatgcgaagtcgttttaacgcatgctttatcgaatataaaatgtagatgggctaatgtggttttacgtcatgtgaataaaagttcagcattcgtttaataatatttcaatatcggtgtctagagacccgtggatttgtatagtcagcaccatgatatgaatctataaaatattgtatctccaattgcaattcaatcgatataagaaattaatacaagccgttcatatagtaaggttgccaatggcattcaataacgaccgtacagttgccgctatattaatctacgtgccatttcttaaataaagataggcgaatgactatcgaaaataaaacaattattaatgagtgaaaacgtattgcacaaataaagattcattatggttggctcaaattttggctctggtgctcgatgacgtcgagatgaggacagtagtgatcaacttggcggtcgataccttggttacgccactcccagagtgccatgtcgtcctccgagcggtctgagataacccagtcggcaattgctgctgcattgccgggcgttccccaaccacgacgaatatgctttcgttcatctaactcgcgtcgcactgccctcccagtcatgaagtcaaagccaaattctaccctctctccatttcccagctcagtcgagaaatcgtaacacctcgtggcagctgacagtttcagaaaggggcgtatccctcgaactccagggtcctctttcacatagttagcaaggcgtactgctgcataatctgcgttgaaggctctgatgactacaggatcctcggacaagcccaattgatcagggcgaaccctcgcgctcataatatgaattgcgacgacccttgcttcctgtcggagcatcgaatcaatccaagccttccctgcggcatagaggtcatcgactgcgatgtcatcaagatcgagtagctttgccaacctaggaagttcttgaggaaaaatcaccggcatgacagcaaccgtctctcgccagtcagttgccggactggcttccctaacgccatccacgaatgcctcaccgcttgcgtatttgaatgtgtaaaagagaaggaccactctttggcggtacttcggacgccggcttagccacgcggcaataatgtgggcctcaaactcacgaccatccaaaaatatagtcgcgcctggattgacctcgctggccttgtcgagaagaggttccaaaaagggaacggtgtctttcgtaatagtacttaaatctgtgagttcgccatgcgaaacctctcgaacgattatcggcgtatccctgacatcagctgaatgaaattctcggacgagtttgtcgggcaaagtggagacccgccacgtgttgaagtcgtgggaaacgatgggcacatcgtcgccggtgagtgcggcatcgagctcagagaggttccgcctgccaacctcaccgagagcagctaacaacgaagtttcggtgcattcctgtatccctttacccagattatacatgccccggtgttcgataacttgaagaggcagtggctcctcaagatgttcaaggaggtggggtacagagtgccgggcgaggacctcatccaccgtgacaccaaccgggagatcccattcgagtttccactggggccagcatgtgcccgcgacggcgaaaggtttgcgctggcaaagaacccggctgctgcaggtggacctatccttacccatggcaatggggttttgctaaaaagtcaggcactttactgggcaattgatagggtgggattgcgttattaactgttctccagcgggaatctttatctttattgaaatgctaaagcacttagataaaatacagctgtaccgcaatataaaatagtaggataatgtaatatgtgtatcgagaatacgacaagctaatataatctagcgtcaaattgcaataatttaaatcaaaactactgatgaaataataaaagatggtcaatttttattggtaggagttgtcgaaagattcgacggacggccattacaatacataggtgcaagaagtaaaacaggaagggaaacggaaaacagtgctataaaaaagcgacagatcgcggcgatcactgactgcgatcgggaagaagctcgccaagttcaccgagaatagcagagagcgcatcctcatcgggtactacgaacacattcgtcccagagggctttgtttcagctgcgccaacccagaaagcaaggccattttccaagttgccgatggcggtcagcatgttttgattgttgctgccgtttccacaagcgatgtgaaggccgatcccgtgagagaggcccttgacgaaggtgaaatagcctttggattttccaactgtttcaacgggcactagatattgaccctctggcgcggcaaccaccttgaatttgcgagatgactggttgccgatgagcgaagaaagcatttctccggcttctttgtaagatttgtgagattcccacatttgacagccgtagaaatgccccatcggaatgttgcggattcccgggatgccaccaaatttgttctccatagccgcgtgaacggcttgccagttgggcagggagaaagaatcgaagcgatcatctttgtagatcgtgaccattccatcatttccctggaatccgatattttcaatggcgctgaaaactgaccttgcgatttcttcgcattcccgtgcggatgtgagcaattgataatggcccttgcaggcgatcctggtcaaattggcgatgatgttgatggcaggattaatatcccaacactggtgatttcgatcttgcttaaaggtggtaccatcgccgtcgaaggcgagcagggcccggagagatgaatcggcaagactgcgtcggacccgctccgcggcgtcgggaatgaggctgataagagacatatccaaaggtgtttgtgggtaacgggctgctcaatgaagccttaaatgcaacgcaacatatgtaaggatgagttgacttattggagagagaaataggaatgagctggccagccattatcaacgtggggccatgctgacaatgtttacgtgaaaggctcaactacctcgaagcagacctctatattcgttgactttattactgaacaagaagttgcttgccactcattttcttaaatcttgccctttctgcgcctcgctatcatgcccgccaacgacgcgacatgcgctgccgcgattgccttccccgagggcaactggaaggaagaacttgatgcgctccgcaccttgtgtgaccccgtcgaggtggttaaggtcgcagtcggcagaggtcttagcggcatatgtaatgttgttgcagcaatgaatcccacaaaggtgaggggcctcggcgatgtcatcgggcagatgccggctcttaatcaccgtattgctgccgccgccggcgaaactccggtgcgagaccttggaataggttaccagtgcgcaatctgccaccccgacatagccagtgcgatgttagccacttctgaggggatcagccacgttctccgtgaaaggattgagaaagaagttgaccgggacattggagaaggcgccaccgtctgcattttcgttcagccgagaatgagctccaagggctctccagtttctgtccatttcaccctccagtttgcgagatctggaactcttgtcgatgccagaatgatggagagttacaatttcatgaaaggcaatggcacagtgaccgcaccggatttgaaaagtcattggaagaagcacggtattgacaggccaggcccacgtccgcccacgtccaagtttgaactcctcttcgccgctgtccccgacaacagtaaacttgccgccaccgattttacccatctcggccctgtcgagcgtgataaggaactactcggcagcacggtattcgggattgccgctaagaaacctggtacgatcgtttatccgtgcgaaaaggttctctgtttggaggtcgacgtacacgcgcatcgcgccctagaagtacttcaccgccttggggaacaggcttatagcaatggccgtggcactagcttcggtcttcacaccggtccgtcctcttgccttaatctttccgccgccgcgctcgctacatttttcaaacgctcggatctctgttcccttccattgagtgatgcttttgtccttttctgcgacccgccaccgcctacagcgccaagaaagatggccttccgatcactgccttctcccccacgagcaccaatcagttcgaactcgtagagcctcaggtcgtcaaggcatatgttctcggacttttcgacgcgccgacgatggttacgccccgcgacaaaacgcgagccagcttctgcagccaatatgtacgtttccgtgaaccgcatccctgtgaagagttcaatgaaattggagttttgatcctcgatgctgctgctaaaatgctcgaacgttatgcaaaatttctagaagatggtggaagagatgatgatgaaatggcgaacataatagatgtatttgggttttgtcttaactagtggattgattgaaacaaaggagtccgagttgggattccctttcggtcttcgtcgtgcaacgatatcgtatgcgtacaggtatcacatttaacgttgctgcggcggaccgagcccgcttggaagcgattgttgcagctccaacttctgctcagaagcacgtgtggcgagcgaagatcatcttgatgagcagtgatggctcgggaacggtcgcgatcatggaggcaaccggtaaatccaaaacctgtgtctggcgctggcaggagcgcttcatgactgagggcgtcgatggccttttgcacgacaagagcagaccgcccggcattgcgccgcttgatggcgaactcgttgagcgtgtcgtcgcactgacgcttgagacgcctcaacaggaagcaacgcactggactgttcgtgcgatggccaaggccgttgggattgcagcctcttcggttgtgaagatctggcacgagcatggtcttgcgccgcatcgctggcgctctttcaaactgtcgaacgacaaggcctttgccgagaagcttcacgacgtcgttggcctctacgtctcgccaccggcccatgccattgtcctgtccgtcgatgagaagagccagatccaggcactcgatcggacgcaaccgggactccccttgaagaaagggcgcgccggcacaatgacccacgattacaagcgccacggcaccaccaccctatttgccgccctcaacatcctcgacggctcggtgatcggccgaaacatgcagcgtcaccggcatcaggagttcatccgttttctcaacgccatcgaggcggaactgccaaaggacaaggccgtccacgtcattctcgacaattacgcgacccataagcagccgaaggtccgcgcctggctggcaaggcatccgcgctggaccttccacttcgtcccaacatcatgttcatggctgaacgccgtcgagggattcttcgctaaattgacacgtcgacgtctgaagcacggtgtctttcattccgtcgttgacctccaggccaccatcaaccgcttcgtcagagagcataatcaggaaccaaagccgttcatctggagagcagatccagacgagatcattgcagccgtcaaacgtgggcaccaagcgttggaatcaatccactagcgtatgaacagtaataagaaaatcccgattgtgaatagtcccaatttcaaatgtgtccgtgtgtaatttgcgtgtcttcagttgaatttcctttaataatatcaaatattcaattgtgaaaagttgtattggttcaggttcaagctttccgaatttgttgaattttattccctgttttcaatttgttgacttgtttgggagacaccttttttgtgtttcgtgaacatgtcaccccttcggtatacattagcctacaaagtaaataacgttgataaatgtcactcatgttgtaataaaattgagcttattatgtataaccagaccctgtgttaatctaattacaaagaaattcatcattctcccaagcaatcctgagtagctgcgtgatggatcttccatatcagcgcccacgtttcaccccgtttgccgtcacccatccacgtagtggagtcaacctgaaccgtgcaatttctcaggcctttgtctgctatgatcagttctgcgaacggctcttgcgatatcagcaaagctggacggattgggtgttcgaccacggatttgcagaagccattgaagacgtggcgctggtgttccaggttgcaccttgccttcatggcccccgaataggcgcgctcgaagtgttgatacctcgtcgcacccaggtcttcatttatatgtcgaacaaccaattgcagcgctttgttgcacaccagtgcattgctcaacttggcgacgccgtgcttgcttgcatgatcccgccctacgcgagtgacctctcgctgcaggaaatggctcgggcgcacaacagattttgcccaggcagttacacgaggtccgcagacgtacagtgctttatcgccatccaactcagcagccgattcgttgaggagggcacatgtaacgtgcacgggcgaaatggcttaaaaagaacctgccgcttctttcgtcgccctgctgagttcttcagccgttatgacatcgttgccattgggccggtgctcttccatgatgaactggattgcccagcaaactgcaatgagcctctttcctgctttgacctgcggtacgactatcaggttttcctccaggagtgcgatgcccatgatggtgtggggcattatccggaaggcgcaccactacctagtgttgccatcgtaggaggcgggctgtctggccttgttgctgccacagaactacttggcgctggcgtcaaggaaatcactcttttcgataccgttgatgagatccgtagttttggggcatcgccgatgccaaacggcgacgctcaccaggccttgacgtcgttcggtgtcatgcctttctccgccaaccaactttgcctgtcatactatctggataagtttagaattccgtccagccttcgttttccttgtgccggcaacgaccacacagcactatatttccgccagaaacgctacgcatggcacgcggggcaagctccgccggggatatttcagcgggtacatgtcggatggaagacactactctaccaagggtgtgaacggaatggcaggagactgatggctccgatggatatctctttcatgttgaaagagcgtcgtcgtgatgaagcctcagaagcacggcagctttggctccgagagttcggaaaattcactttccatgccgttttggtcgagatcttcagctgtggtaattcgagtcctggtggcaaggcatggcaaacaccccatgatttcgaggctttcgggatactgaggttgggatacggccgagtttcgtcctattacaacgtgttgttttcaacgatcctggactggattatcaatggctacgaggaggaccagcatctttctattggtggggttcaacttttgcaggctctgatgcgcattgaaatattccagaaaagccatgcgaaagcacgactctgttttgatcccgtgcgtggaatagccaaggagggcgggagattgaaggtatgcttgaaacacggtcattcgcgtgtttttgaccaggtcatcattggcggcagtgctgaggccgctacagttgataacagactggccggggatgagacttccttcagctacaatatcgaacccgccgtcggaaactcgtctgccgctgtcaattcagcactcttcatggtcacgaagcaaaagttttgggttaactccggcatcccagcagtgatatggaccgatgggcttgtccgtgagctgtgttgcattgacatcgaatcgccagctggagagggccttgtcgtttttcactatgctttggatgactatctatcccggccgatcgagcatcatgacaagaagggacggtgcttggaattggtcagggagcttgctgctgcctttcctgaactggcttgtcacctggtcccagtcaacgaagactacgaacgatatgtcttcgacgaccacctaacggatggttttaagggagctttgtggagggaaaattctctggaaaaaggtcagtatatccaggatctgcctgggaataattttcctattggggatcacgggggagcctatctgattgaccgtgacgactgcgtcaccggagcctcgttcgaggagcaggtgaaggcgggcatcaaagcggcctgcgccgtcatccgcagcaccggcgggacgctctcttcactccaaccggtggactggaataaaaaatagaaatttcctgattaagttatagtcaatgtactattgcgtgttaatcccgtaggtatgcaagctgcaccggcagcatcataatttgatgttccatcaataaattaaggtgcccgttcattgtgtattacattatgtatgtttatcaaaaatataatcgaagtccattttaagtctgatattaattggaattccaaacgattccttgatgcctatcttcgctatgattgtatggtaataaagtctccacatctcccgaaaaatgctttcgtgatttacttgtctctcacgtgctttcgcatcttgacagccaaaagtgggcaacttgagaagagtattaactggccacgcaactcgagatattcccactaaccccaatgacgtcattgcactcgtcacgggtagcagccccacttgcctttgccactttattaattctttggcccactggccattaattggcacctacatatattagtggagaagataaagtgtcactatcgtttcctgttcaattttgaattttgcaaggatttcatgttgtcaactacacagcttgaaaggaaatccgcaatcaacggagaaacgtcaacatctcgacaaaaaaagaatgcttcatcattgcgtagactgcatattgaccgctcctttcggcgctgggcctgcttttactgttgcctagcgttcggacagccaccagagaatgggctatatagatcctttcatcaaaccaaaacattactaagatcatgctgtaacgcttcaatacggtgagtgtggttgtaggttcaattattactatttttgaagctgtgtatttccctttttctaatatgcacctatttcatgtttcagaatggaattagccggactaaacgtcgccggcatggcccagaccttcggagtattatcgctcgtctgttctaagcttgttaggcgtgcaaaggccaagaggaaggccaaacgggtatccccgggcgaacgcgaccatcttgctgagccagccaatctgagcaccactcctttggccatgacttcccaagcccgaccgggacgttcaacgacccgcgagttgctgcgaagggaccctttgtcgccggacgtgaaaattcagacctacgggattaatacgcatttcgaaacaaacctacgggattaatacgcacgtggctggcggtcttcgattcatttccacgccggagatgatatcgaatatgttctgttaagttaaaataagctgcgagccatggcgcgattgtcctgttttattaatatagtactttaacgtctctttagagcgtttgtgtaatgtcgtgaaaatgttttatgtcaaatgtactgttgaactataatattataagtccaggtgtgtcgttgttgttgatactgcaatatatgtgtagtagattagatagtcatatgagcatgtgctgtttttggcaaaattcagcagcaggatcaacacagaagaaaatatttagtacaagaaaataggtcaacacattacaacgtacgctacaactcccaaggttctgtgtcacagactgcgggagggtacatagaacttatgacaaactcatagataaaggttgcctgcagggggagttcaagtcggctttaggcttctttcttcaggtttactgcagcaggcttcatgacgccctcctcgccttcctgatcaggccccgagagtcgcagggttaggtctggctccggtgaggaggcggccggacgtgatatcccgagggcatttttggtgaattgtgtggtgccgcaagctacaacatcataggggcggttttcagtccctcgccgcagaaagaaggtgcaagctacctctctcccgtaaacgttggtcacttttaactccagcaagtgaatgaacaaggaacttgcgaaaatggcgatgaagcattctaaatcaggttcctccgtgcggctgtgcggccaagcaaggttgtgaacacggagcatctcctggagggcgagctcgctccgatatggttgaatcgttgtcgccagcacggcctccattccaaatgtaatggattgttccttcagcactttctgcatcttctcgcgagaaagatagacaaatacatgttggtcgttttctcgagccagatccggctgactaacaaacataggaggatgatagcagactttgttcttcaagagctcagctagttgtttaagtatatatatcggtggagagttttccttcaaatctagcactgcaagagcccatcgtttctggaaatgcaggaggggtttgctatagtcacggctatagattgcaaaagcaaatcggatcccctcgaataggtttatctggctccatgctggagtgagatctactggttgaaatcgtggaaggaatagcaatttgggatccattgtgatgtgagttggatagttacgaaaaaggcaagtgccagggccatttaaaatacggcgtcggaaactggcgccaatcagacacagtctctggtcgggaaagccagaggtagtttggcaacaatcacatcaagatcgatgcgcaagacacgggaggccttaaaatctggatcaagcgaaaatactgcatgcgtgatcgttcatgggttcatagtactgggtttgctttttcttgtcgtgttgtttggccttagcgaaaggatgtcaaaaaaggatgcccataattgggaggagtggggtaaagcttaaagttggcccgctattggatttcgcgaaagcggcattggcaaacgtgaagattgctgcattcaagatactttttctattttctggttaagatgtaaagtattgccacaatcatattaattactaacattgtatatgtaatatagtgcggaaattatctatgccaaaatgatgtattaataatagcaataataatatgtgttaatctttttcaatcgggaatacgtttaagcgattatcgtgttgaataaattattccaaaaggaaatacatggttttggagaacctgctatagatatatgccaaatttacactagtttagtgggtgcaaaactattatctctgtttctgagtttaataaaaaataaataagcagggcgaatagcagttagcctaagaaggaatggtggccatgtacgtgcttttaagagaccctataataaattgccagctgtgttgctttggtgccgacaggcctaacgtggggtttagcttgacaaagtagcgcctttccgcagcataaataaaggtaggcgggtgcgtcccattattaaaggaaaaagcaaaagctgagattccatagaccacaaaccaccattattggaggacagaacctattccctcacgtgggtcgctagctttaaacctaataagtaaaaacaattaaaagcaggcaggtgtcccttctatattcgcacaacgaggcgacgtggagcatcgacagccgcatccattaattaataaatttgtggacctatacctaactcaaatatttttattatttgctccaatacgctaagagctctggattataaatagtttggatgcttcgagttatgggtacaagcaacctgtttcctactttgttaacatggctgaagacgacctgtgttctctctttttcaagctcaaagtggaggatgtgacaagcagcgatgagctagctagacacatgaagaacgcctcaaatgagcgtaaacccttgatcgagccgggtgagaatcaatcgatggatattgacgaagaaggagggtcggtgggccacgggctgctgtacctctacgtcgactgcccgacgatgatgctctgcttctatggagggtccttgccttacaattggatgcaaggcgcactcctcaccaaccttcccccgtaccagcatgatgtgactctcgatgaggtcaatagagggctcaggcaagcatcaggttttttcggttacgcggatcctatgcggagcgcctacttcgctgcattttctttccctgggcgtgtcatcaagctgaatgagcagatggagctaacttcgacaaagggaaagtgtctgacattcgacctctatgccagcacccagcttaggttcgaacctggtgagttggtgaggcatggcgagtgcaagtttgcaatcggctaatggttagtcgatgggctgacgagtttgatgtcaggagaagctgagtgtgtcacttgtttccctttaagaagtattaatgtaataaaaatcaagatctggtttaataactggatacttgatttcatcgcgctttttttgaataaatgtttgttgtcttgactttaagatatcctttgaaatttgcgttattcgtatttcgcttttggttatttccaaaagactttgctcagtaagatcaaacgtttgtatttctccgggccacaatatttgacctatatgcactggcccacgcgccgcaatagatgaaaattgccaaaattagctatcggtcttctgaaaagaagggccgacatgttttcatagaccatgcaaagtcatactacctgaaactgataaataacgacaaagaaagtagcctatttaaaagtcgctatagcatgaattcaacacaaggaaaccaaaagtcggaaggaagactttaatcccggattatttggacatgataggagctatggggcaacgtgtcattttcatgagtgttgaatgattttctgtagcaaatagaaaacgttttttaaaacgatgtggccttggagtaatcagcggaagaaatggtcatgctcagataatttccgttgctgacctcgcaaccaacccctttaaatacctctgctgcccatgcattttgccaagttaacctaaagtggcagctgaatggctcgttattgcagtggtggctctcaacggcttcatgtcgatgattttcgttggatcaaggagcccactcgactgaaggctcagcttattaatgtggtggagacctacaaggctgcacaaacagagacgttaaagtactatatatcatctgcaactgagcgtgtggctcatgtggaggcagccgaggtcaacaatgcggaaatggagctgcatcctgctgggttgaagtaccctctgtccttcgtctttacctccctggccgtggctacagcctgcaaggagaacaagcatctcttgtgcgaggagcatttggagggggacttgatatcgtgcgtcgttcctccctatcagacaaatgtctcactcgctgctttaagggagctccacaattccatttcgggaggagggtaccaggaacaagcagacatggattattttgtggcgatcatcccaaatgataatttcgactatcagagctgcgaaatcgacacacgaagttgcggtaaaggactttgcaagatttatagtagggaactgggagggcagcctctagcttatgacgccatactggcaatcggcaaggtgctgctgctggaatagatagtgggccgctgatccgagtttgattttgtcgtattatgttacgtgaactttttatcatgcatgtttcgcttatgctcccgagtgtcggccatgttgttgtgttaaaataaaaggctgatgttaagtcctattgtaaaatacctttatagattaaatatatatagtataacttctgtatgccgtcgatgagcggttatatgattgtaatctatacgttgttgcaatcaatcgtattacagtgagccgtgcttaatgaaataaacatcatgttaaatgtctatttattcaatcaacatgcgctgacaataatcaaaaggggaaacgtaataacattgcggtggatacagcgtttattgggaggtccgcgggccgatacacttaaataacatagacagaatttgagagagcacgcaggttgtagccaagttgagcgacttgccggtagcacggaagctaagctcaggtgttacaaatagacaggcgtcgaggcgacgagcacgacgaccttgccggacattgcggtcgcagggggctcaaagcggttggcttgtaacggaccttgtgtttcttgttgtagctttcatcgagcataaccattgggacggttgctgaacaacggtaacgcacttttttcacgggagcgaggtagaagaacatatttccccgtcggcagccggcggtgagcatgccaattcctaagggatcaatggactcgtgcgaacggtgagcatgccgttctgaccgtcggtgcccaatcagcaggccactcccaacatgttttccaagtccttaaaaccagtctttatagcattgatctcccagcaatctttattgaagtcgattttaatattcaaaagaagattttagtggaaagggaatataatcgcgtggccgaagaagagccttcaaaaatcagaatccactaggataaacaataatatctgaaaagcattgaatttgggttaggcacgagaggctgacgcggatgccactcgattgctagtggaaggattcccttttttctagcgtatcgaattcaccgtttcactatatgttttcctgattggttgatctgcgggaccaccattgactgccactaatatcgaaagtgggtctgcttcgattatgatgctttgtgagaggttctcttcccaatgcatgcaagctggcagattcggatactctcaatagagatcttatttcgcgtctcaaaaagttcccagaaatcaacaaaggggagggcaggtcctttaaatacgttgcagctgtcctttaaaatagaagagaatttacagctggaggcacagaccactaaactgcgaaagtaagcatggcagatgagttggagcgtcaattggaagccatttctctcattacagtcctgggtccggatgtgaaggctgagcttgaggcggagctacgagactactgcgaagatctcgacttctggaaaagccacggtttaccggtggcggatctcgatcagactgtgactgtcgacaagcttctatacatgtatatggatcgggcaacagcagacctgtgtgtgaagaatcgctgcctcgtttgcaacagtggcaattcagccgcaaaagtaacctcgcttccaccataccttgcaggcgtgacaagcgccgaggcctatgagaaactcaactccattgttgatgggagtgtcgccccccaatctcgtgggcctccctgctattttgtggcgttcctgcccagcagctgtttcgagaaaaccagtgagatatcggtgcgcacagtggacggcgagtgtggccccttcgatgtctttacccggcagcgtcagccacaggatcagagtgatatgttttttaaatatgaaggagttgtatgtgctggaaagagtgtatttatgtaagaattatcttttatagcctgtgttacgtttgaacccggtccgcgcggtattgttttcaataaatggtatgtgcggaggatataattggtctttcattggtgtgatttacgtgtaacgcggataataataaagtaaattacaaaagagaaacgcataattttattccagaatgattgcgagaaacgatgaaaatacatgaaaatgcatattgtcgccagggaaggatggcgccgaaataaacgaaactgagccaatacagtgacttgccaagcgagtttgatcctaccaaattcgcgcaaattaatgcccgtgttccatcgggccagcgagtttattcaaaagagtttcgtacacgtgggcggcgacggcaacgtcaatgcttgctagccctaccggcgagaagttggccggccccttccatgccttgaggtcattcatcaaggcctcgtcatcgagaatttcggtgtagttcttgatcccatcgcgcttgccgtgttgggtcagtttcataccgcgcctagaatagtagagggcaacggcatcaacgttgcgggcttccatcgcaacaaggtcatcggcgacaattagaccatccgcagataggacatgctcaatgtaatccggcggcatgtcatcaataccgagtgacaaagtgactgcgttgggggcgatttcagcggcttcgaataccggttttccgtagttggtcgccatgatgacgaattgagaatatggcaaaaggctacgatcgccgacagcttcaaggctaaaggttacgcaatcacgtaacttttcgacgagctcgaaattggatttcttaccgcggctgagcactgctaccttacgaattctcttagcggcaccatagttaagtgagagaattacagcttcggcaacttttccagccccaaacaagaaaacgtcgatgtcctctctgccttgcaacagcaggtttacgcatgctagcgagaaccaacccgttcttccattagaaattgccacgccctctaccgacataaggagcgtcccggacaccttgtcgcgcaggaaaatatcggagtgctggagcggctttccggtagcggcgttggttggcgcgaagtggatgtctttggtgccggaatatcttccgaaatagccaatgagtgctccttcagtccatccaggaacattcttgttgaacgttaggtaagctttgacatgtccggcttttcctgcggcaaacacctcccaataggacttgagagcttcgtcaacaaatgctggtgtgatctggatatcgaggtttgatagtgcagattcagtccagtgtacctcgcaaagttgtttggccatctgccttgtaggtgcgaattttctctgctcaaattgttgaggttagcggatttgtaaacgcgtttatatgggctgcttggagggtacttttggattaatttttttctgccagcgcattctgacgcggcaccgctttggaaagtgcgctgtgggtccgcgttttctacaataatgtgccgatccggtcagaaagtatatggatgagttgtgccagcctcaccaacgtgctgcaggcccatcatgactacttcaatgttaatgggggtaatgaataaataggcgaaattgggttcacggtgggcccagggaatataatattgccgcagaggtagtcggatgccaaggcccgcaactaatagttcacgaacaaattcattgtagtgggcggccaactccaaaaccaattgccagttattgtattgcaatacatatatgagtattcggatacaactaatttcattaaataatattttaagtgtggacagaatagcgcctaataaatttgcgaatgttgtccaattgacgtttttataggtaactcgataaatcgtgcttttgtgatattctgatgcggacaatatacatttaaacataaagatataagttattgaggcatttatgtatattacaatagtggggtacatttttcacagatgctgtcacccatgaaatattggcaaaatactcttaaaatatgcaagaaactaaagaggatgcatgggttgggctgtaggtacatggatgcaaatgctgttttgcaataagtcatatagtctcgtctgttgagtgaggcccattcaatcagcaagtaggactgaggtgcatgatcgacatatttttgaaccacagttttggcaagtttttcatacaaatgcacggctacggccaaatcgtagcttgcaagtccaactgctgaaaagttagccggcccgttccaagaaattagcctttgcataaggactggatcgcggagaacttcagagtagttcctgatcccattgtccctgccgtgttttgttagctttaaatggcgtcttgaatagtgcagcgccaacgagtcgatattacgtgtttccatcgcatccatatcatctgccaccacgatgccactcagcttcaacacgtgatcaaaatagtcagctggcaattcgtcaattccaagcgtcaatgtaacggcattgtctgtgatctccttcatctcaaagacgggcttgtttgaattcgtcgccgtaattatgaacttggatttgctgagatatgctcgattgttaacagccttgagtgaaatcttgacttccggctgaagcctttgcaccaactcatggtttgactggttgcagcggctgagaatcgcgattcgttgaattcttccagatgctcccgaattgagggcgaggatgatggcctcggcaactttacctgctccgaataggaagacattgatctggcttcggccctgcaataggagattcaggcatgctagtgccagccaaccagttctcctctccgatatagccaccccatcaacagagaagagacgtctacctgtgaaacgattgcgaagccaacgtcgatgtgagaagtcggttctttgtatctcgcgtttgacggattagaatggatgcttttcacacccgaatagtcgccgacgaaacccaccagagctccctccgtacagccctctcgatcaagtggaacgaagaccttgttgtggccgagccgcccttcagcaaagaggtgccaataatctttcaaggcatccgcgacgagttccggtgtaatgtatattccaaaagccgatagagattcctctgtccaacattgctcgtgtatttgatcggccatgtttgtgtttgatcagcctcctttcgaaaatttcttgagtttcgaataattctaaaatcgaaggacgattaatagtgccataccaagacaagaagggtaggtgggccatcaatccacaagcctagcacattttgctgtctgctcatgcaaggtatccaatggaagcctggattggttagccgaacttggtgggttcaattggagcgggcaggtcactttttgtctctcaaataactgaaactaagttttgttatttggtatgtgtttgtctgttctgccgaaggtgcccgaatttgcgcaaattcctttctaaaaaggcttacatctagcaaaaggtgagccctgtgcatcccagcatttggacaaagcgcgccaattcggacagcgactggctgcgttggaggctcggatctcaaagaatagaaaagagttatgatcatgttcagaaccgccaattttgtgcggtatgagctctttgatgaaagtaatggtttcaaaaaagcaacatcgtgggtgaaaggtacctacatatcttcacagacaataactactgttgctgtttgctgattgactgacaggatatatgttcctgtcatgtttgttcaattgttcaattgttcaattgttcaattgttcaattgttaatgtataagttcgtgatgaaggatggttgttttaaaaatagtatgtttgactgaggttaagtcactcacgttttgcacatcgacggaccgtaagcattctttcggtaagaccgaagctcgtcccagataataggccccgtggagggaggccttgtatgggccgaccgatgggcgtgctgagccgagtacggcgacgcctgcggcgattgcgcgggcggcactgcgcgcaggggcacgggttcatacgaggacgagcgtaaggggcatagagctttccgcccgtcgggtttcagccatattgcttgattgcggccgactggaatgcagccaggtcgtgctcgccggcggcgcctggtcgagcggcatgctgcgcacctcagtgttcggcttcctcagctcacggttactgtgtcggcgctaagaaccgaggcctttgatggcggacctggccttttcaatcaagggatgctgattttccatccgtaagcgtctcgacggtggttacaccgtcggcttcggcgcgacgatgagaaccgaaatcgtcagcgatagctttcgttttctgtcggattatttcctcctgatgcgagaggaatggctttctatccggctgccggcgggtgcgcgcgtccggattgaccctcccgttcgttgctacttggctcgagtgacgaaatagcacgcctgtgccgctgtatcatgtccatcgggctcacaggagattcgctcgtagcgcgttggtgtcactcaccaacacgcgtcgtcgcaccaaattggggaggatggtagcggaatcctaaaatcctaaaaccataccgacgcgtcacggcgctcgtgacccctgcgagcgacgcggcactctctcacctgatccgtgctgcggttgctcaatacgcaatgagcattgtcacggttctcagggtaaacggcaatctcttcgtcatgcgggcgtggatgctatcaccgttagaaagggcctgcccccatggtgggtctctaaggttcagtctgagaaggggcagccagagcggcactgtttgaagagcagtctgaaccgctcagatcgctcgcatcgatgcttgggcggcg

(19) United States
(12) Patent Application Publication
(10) Pub. No.: US 2004/0016016 A1
(43) Pub. Date: Jan. 22, 2004

(54) Compositions and methods for improving plant performance

(76) Inventors: S. Luke Mankin, Raleigh, NC (US); Oswaldo da Costa e Silva, Rheinland-Pfalz (DE)

Correspondence Address: SUTHERLAND ASBILL & BRENNAN LLP, 999 PEACHTREE STREET, N.E., ATLANTA, GA, 30309, US

(21) Appl. No.: 10/465,008

(22) Filed: Jun. 19, 2003

Related U.S. Application Data
(60) Provisional application No. 60/389,982, filed on Jun. 19, 2002.

(51) Int. Cl. A01H 1/00; C12N 15/82

(52) U.S. Cl. 800/278

(57) ABSTRACT

Provided are compositions and methods for improving plant performance including transforming a plant with an isopentenyl transferase, a tryptophan monooxygenase or an indole acetamide hydrolase polynucleotide. Preferably, the plant is transformed with an ipt, iaaM or iaaH oncogene from Agrobacterium tumefaciens operably linked to a developmental stage-preferred promoter or a tissue-preferred promoter. Controllably expressing one or more of these polynucleotides in a plant results in increased drought resistance, increased root mass, increased seedling vigor or modulated branching.

### CROSS REFERENCE TO RELATED APPLICATIONS

The present invention claims the priority benefit of U.S. Provisional Patent Application Serial No. 60/389,982, filed Jun. 19, 2002, the entire contents of which are hereby incorporated by reference.

### BACKGROUND OF THE INVENTION

1. Field of the Invention

This invention relates generally to compositions and methods for improving plant performance comprising transforming a plant with an isopentenyl transferase, a tryptophan monooxygenase and/or an indole acetamide hydrolase aggressive.

2. Background Art

The Ti-plasmid of Agrobacterium tumefaciens has long been recognized as a natural vector for the transfer of DNA to plant cells. However, transformation of a wide range of dicotyledonous plants with Ti-plasmids causes neoplastic transformation, or crown gall formation (Nester, E. W. and Kosuge, T., 1981 Annu. Rev. Microbiol. 35:531-565). Appropriately, the “Ti” label designates that the plasmid is “tumor inducing”.

The tumor inducing nature of the Ti-plasmids derived from the pathogenic gall-inducing strains of Agrobacterium can be attributed to the presence of T-DNA oncogenes. Three such oncogenes are ipt, iaaM (tms1) and iaaH (tms2). The ipt gene encodes an isopentenyl transferase that converts adenosine monophosphate into isopentenyl-adenosine-5-monophosphate, the first intermediate in cytokinin biosynthesis. The iaaM and iaaH genes encode tryptophan monooxygenase and indole acetamide hydrolase, respectively, which convert tryptophan to indoleacetic acid, an auxin.

In an effort to remove the tumor-inducing properties of the Ti-plasmid while retaining its transformation abilities, disarmed Ti-plasmids, or plasmids lacking the T-DNA oncogenes described above, were created. These disarmed Ti-plasmids were then used to shuttle desired traits into plants including tobacco and canola. See, for example, Bevan et al., 1985 The EMBO J. 4(8):1921-1926; De Block et al., 1984 The EMBO J. 3(8):1681-1689 (tobacco); and Zambryski et al., 1983 The EMBO J. 12(12)2143-2150 describing the use of disarmed Ti-plasmids to transform tobacco and U.S. Pat. Nos. 5,463,174; 5,188,958; and 5,750,871 describing the use of disarmed Ti-plasmids to transform canola.

Although associated with tumor formation, some researchers have attempted to retain and express the ipt T-DNA oncogene in plants. For example, the ipt T-DNA oncogene has been used as a high-efficiency marker for plant transformation in a dexamethasone-inducible system (Kunkel et al., 1999 Nature Biotech. 17:916-919). The ipt T-DNA oncogene has also been placed under the control of regulatable promoters in order to study the effects of cytokinins on plant biology. These promoters include inducible promoters such as a heat shock promoter (Medford et al., 1989 Plant Cell 1:403-413), a light-inducible promoter (Redig et al., 1996 Plant Physiol. 112:141-148), a copper-inducible promoter (McKenzie et al., 1998 Plant Physiol. 116:969-977), a tetracycline-inducible promoter (Fais et al., 1997 Plant J. 12:401-415; Gatz et al., 1992 Plant J. 2:397-404), and a senescence-specific promoter (Gan S. and Amasino R. M., 1995 Science 270:1986-1988; Jordi, W. et al., 2000 Plant, Cell and Environment 23:279-289). Importantly, however, none of these references demonstrate or disclose expression of the ipt, iaaH or iaaM T-DNA oncogenes under the control of a tissue-preferred promoter. In addition, iaaH or iaaM have not been expressed under the control of a developmental stage-preferred promoter. These prior art references additionally fail to teach increased root growth associated with transgenic expression of the ipt, iaaH or iaaM T-DNA oncogenes. Rather, several of the prior art references teach either no difference in root mass or a decrease in root mass following transgenic expression of ipt in a plant (McKenzie et al., 1998 Plant Physiol. 116:969-977; Medford, J. I. et al., 1989 Plant Cell 1:403-413; Smigocki, A. C., 1991 Plant Mol. Biol. 23:325-335; Van Loven, K. et al., 1993 J. Exp. Bot. 44:101-109).

The prior art has not described the use of an isopentenyl transferase, a tryptophan monooxygenase or an indole acetamide hydrolase for the improvement of transgenic plant characteristics such as increased root growth, increased drought resistance, increased seedling vigor or increased or decreased branching. There is a need, therefore, to identify isopentenyl transferase, tryptophan monooxygenase and indole acetamide hydrolase compositions that can be used for the improvement of a plant in the aforementioned ways. Also needed are methods for improving a plant's root growth or drought resistance, modifying a plant's branching or increasing a plant seedling's vigor that make use of the naturally neoplastic Ti-plasmid oncogenes.

### SUMMARY OF THE INVENTION

This invention fulfills in part the need to identify new, unique expression systems that can be used for the improvement of transgenic plant characteristics. The compositions of the present invention can be used to increase a plant's drought resistance or root mass, increase a plant seedling's vigor, or increase or decrease a plant's branching. In particular, the present invention provides a method of improving plant performance, comprising a) transforming one or more plant cells with an expression cassette comprising a nucleic acid encoding an isopentenyl transferase, wherein the isopentenyl transferase nucleic acid is operably linked to a developmental stage-preferred promoter or a tissue-preferred promoter; and b) generating a transgenic plant comprising the expression cassette from the one or more plant cells; wherein said developmental stage promoter is not a senescence-preferred promoter. The present invention also provides a method of improving plant performance, comprising a) transforming one or more plant cells with one or more nucleic acids encoding a tryptophan monooxygenase and/or an indole acetamide hydrolase, wherein the tryptophan monooxygenase nucleic acid and the indole acetamide hydrolase nucleic acid are operably linked to one or more tissue-preferred promoters; and b) generating a transgenic plant comprising the tryptophan monooxygenase nucleic acid and/or the indole acetamide hydrolase nucleic acid from the one or more plant cells. Preferred isopentenyl transferases, tryptophan monooxygenases, and indole acetamide hydrolases are encoded by T-DNA oncogenes, or derived therefrom. In a more preferred embodiment, the isopentenyl transferases, tryptophan monooxygenases, and indole acetamide hydrolases are encoded by Agrobacterium tumefaciens T-DNA oncogenes.

Expression of the isopentenyl transferases, tryptophan monooxygenases, and indole acetamide hydrolases is controlled by a tissue-preferred promoter, an organ-preferred promoter or a developmental stage-preferred promoter. Preferably, the tissue-preferred promoter is a root tip-preferred promoter or a meristem-preferred promoter and the developmental stage-preferred promoter is a germination-preferred promoter, and more preferably, an early germination-preferred promoter. In a preferred embodiment, the tissue-preferred promoter is derived from a rolB promoter, and more preferably, a rolB promoter from Agrobacterium rhizogenes. In another preferred embodiment, the developmental stage-preferred promoter is a GA4H promoter, and more preferably, a GA4H promoter from Arabidopsis thaliana.

Also described herein are isolated expression cassettes comprising an isopentenyl transferase, a tryptophan monooxygenase, and/or an indole acetamide hydrolase polynucleotide operably linked to a developmental stage-preferred promoter or a tissue-preferred promoter. In one embodiment, the expression cassette comprises a tryptophan monooxygenase polynucleotide and an indole acetamide hydrolase polynucleotide operably linked to one or more tissue-preferred promoters such as a meristem-preferred promoter. In yet another embodiment, the expression cassette comprises an isopentenyl transferase polynucleotide operably linked to a tissue-preferred promoter, such as a meristem-preferred promoter or a root tip-preferred promoter, or a developmental stage-preferred promoter such as a germination-preferred promoter. The present invention includes transgenic plants, plant cells, plant parts and plant seeds containing the expression cassettes described above and below.

Also described are methods of improving a plant's performance, increasing a plant's resistance to drought and/or increasing a plant's root growth comprising transforming one or more plant cells with an expression cassette comprising an isopentenyl transferase polynucleotide, a tryptophan monooxygenase polynucleotide, and/or an indole acetamide hydrolase polynucleotide operably linked to a root tip-preferred promoter and generating from the one or more plant cells the transgenic plant. Preferably, the expression cassette comprises an isopentenyl transferase polynucleotide and the root tip-preferred promoter is derived from a rolB promoter.

Included within the present invention are methods of modulating branching in a plant including the steps of 1) transforming one or more plant cells with an expression cassette comprising an isopentenyl transferase polynucleotide, a tryptophan monooxygenase polynucleotide, and/or an indole acetamide hydrolase polynucleotide operably linked to a meristem-preferred promoter and 2) generating from the one or more plant cells the transgenic plant. Plant branching can be increased wherein the expression cassette comprises an isopentenyl transferase polynucleotide operably linked to a meristem-preferred promoter as described above and below. Increased branching results in increased flowering and seed set. Plant branching can be decreased wherein the plant is transformed with both a tryptophan monooxygenase polynucleotide and an indole acetamide hydrolase polynucleotide operably linked to one or more meristem-preferred promoters as described above and below. Decreased branching reduces tillering, a trait that is particularly desirable in maize.

Methods of increasing a plant seedling's vigor comprising, increasing cytokinin levels in a plant seed during germination and generating the plant seedling from the plant seed are also described herein. These methods include the steps of 1) transforming one or more plant cells with an expression cassette comprising an isopentenyl transferase polynucleotide operably linked to a germination-preferred promoter and 2) producing a plant seedling from the one or more plant cells. Preferably, the germination-preferred promoter is a GA4H promoter from Arabidopsis thaliana.

### BRIEF DESCRIPTION OF THE DRAWINGS

FIGS. 1(A-B) show the nucleotide and amino acid sequences of an isopentenyl transferase from Agrobacterium tumefaciens.

FIGS. 2(A-B) show the nucleotide and amino acid sequences of a tryptophan monooxygenase from Agrobacterium tumefaciens.

FIGS. 3(A-E) show nucleotide and amino acid sequences of indole acetamide hydrolases from Agrobacterium tumefaciens.

FIGS. 4(A-C) are schematic representations of several expression vector constructs included in the present invention.

FIG. 5 shows the nucleotide sequence of the TL-DNA region from A. rhizogenes, agropine-type plasmid containing rolB promoters.

### DETAILED DESCRIPTION OF THE INVENTION

The present invention may be understood more readily by reference to the following detailed description of the preferred embodiments of the invention and the Examples included herein. However, before the present compounds, compositions, and methods are disclosed and described, it is to be understood that this invention is not limited to specific nucleic acids, specific polypeptides, specific cell types, specific host cells, specific conditions, or specific methods, etc., as such may, of course, vary, and the numerous modifications and variations therein will be apparent to those skilled in the art.

Described herein for the first time are compositions and methods for improving plant performance through the expression of an isopentenyl transferase polynucleotide, a tryptophan monooxygenase polynucleotide, and/or an indole acetamide hydrolase in the plant. As used herein, the term “improving plant performance” includes, but is not limited to, increasing the plant's resistance to drought, increasing the plant's root growth, increasing or decreasing the plant's branching and increasing seedling vigor.

The invention provides a method of improving plant performance, comprising: a) transforming one or more plant cells with an expression cassette comprising a nucleic acid encoding an isopentenyl transferase, wherein the isopentenyl transferase nucleic acid is operably linked to a developmental stage-preferred promoter or a tissue-preferred promoter and wherein said developmental stage-preferred promoter is not a senescence-preferred promoter; and b) generating from the one or more plant cells a transgenic plant comprising the expression cassette.

Preferably, the isopentenyl transferase nucleic acid is an Agrobacterium tumefaciens T-DNA isopentenyl transferase nucleic acid. More preferably, the isopentenyl transferase nucleic acid comprises a polynucleotide sequence selected from the group consisting of: a polynucleotide of SEQ ID NO:1; a polynucleotide encoding a polypeptide of SEQ ID NO:2; a polynucleotide having at least 80% sequence identity with the nucleotide sequence of SEQ ID NO:1; a polynucleotide encoding a polypeptide having at least 80% sequence identity with the polypeptide of SEQ ID NO:2; and a polynucleotide hybridizing under stringent conditions to the nucleotide sequence of SEQ ID NO:1.

Expression of the isopentenyl transferase nucleic acid in a seedling derived from the transgenic plant is useful for conferring increased vigor in comparison to a control, wild-type seedling. In a preferred embodiment, the isopentenyl transferase nucleic acid is operably linked to a developmental stage-preferred promoter, wherein said developmental stage-preferred promoter is not a senescence-preferred promoter. Preferably, the developmental stage-preferred promoter is a germination-preferred promoter. More preferably, the germination-preferred promoter is an early germination-preferred promoter. Most preferably, the germination-preferred promoter is a GA4H promoter from Arabidopsis thaliana.

In another embodiment, the isopentenyl transferase nucleic acid is operably linked to a tissue-preferred promoter. Preferably, the tissue-preferred promoter is a meristem-preferred promoter. Such expression is useful for producing increased branching in comparison to a control, wild-type plant. Preferably, the plant is a Brassica napus plant.

In another preferred embodiment, the tissue-preferred promoter is a root-preferred promoter. Preferably, the the root-preferred promoter is a root tip-preferred promoter. More preferably, the root tip-preferred promoter is a deletion derivative of a rolB promoter from Agrobacterium rhizogenes. Such expression is useful for conferring increased drought resistance in comparison to a control, wild-type plant.

In another aspect, the invention provides a method of improving plant performance, comprising: transforming one or more plant cells with one or more nucleic acids encoding a tryptophan monooxygenase and/or an indole acetamide hydrolase, wherein the tryptophan monooxygenase nucleic acid and the indole acetamide hydrolase nucleic acid are operably linked to one or more tissue-preferred promoters and are expressed in the one or more plant cells; and generating from the one or more plant cells a transgenic plant comprising the tryptophan monooxygenase nucleic acid and the indole acetamide hydrolase nucleic acid.

Expression of the tryptophan monooxygenase nucleic acid and the indole acetamide hydrolase nucleic acid in a plant can result in decreased branching in comparison to a control, wild-type plant. Preferably, the plant is maize.

In a preferred embodiment, the tryptophan monooxygenase nucleic acid is an Agrobacterium tumefaciens T-DNA tryptophan monooxygenase nucleic acid. Preferably, the tryptophan monooxygenase nucleic acid comprises a polynucleotide sequence selected from the group consisting of: a polynucleotide of SEQ ID NO:3; a polynucleotide encoding a polypeptide of SEQ ID NO:4; a polynucleotide having at least 80% sequence identity with the nucleotide sequence of SEQ ID NO:3; a polynucleotide encoding a polypeptide having at least 80% sequence identity with the polypeptide of SEQ ID NO:4; and a polynucleotide hybridizing under stringent conditions to the nucleotide sequence of SEQ ID NO:3.

In another preferred embodiment, the indole acetamide hydrolase nucleic acid is an Agrobacterium tumefaciens T-DNA indole acetamide hydrolase nucleic acid. Preferably, the indole acetamide hydrolase nucleic acid comprises a polynucleotide sequence selected from the group consisting of: a polynucleotide of SEQ ID NO:5; a polynucleotide encoding a polypeptide of SEQ ID NO:6; a polynucleotide having at least 80% sequence identity with the nucleotide sequence of SEQ ID NO:5; a polynucleotide encoding a polypeptide having at least 80% sequence identity with the polypeptide of SEQ ID NO:6; and a polynucleotide hybridizing under stringent conditions to the nucleotide sequence of SEQ ID NO:5. More preferably, the indole acetamide hydrolase nucleic acid comprises a polynucleotide sequence selected from the group consisting of: a polynucleotide of SEQ ID NO:7 or SEQ ID NO:8; a polynucleotide encoding a polypeptide of SEQ ID NO:9; a polynucleotide having at least 80% sequence identity with the nucleotide sequence of SEQ ID NO:7 or SEQ ID NO:8; a polynucleotide encoding a polypeptide having at least 80% sequence identity with the polypeptide of SEQ ID NO:9; and a polynucleotide hybridizing under stringent conditions to the nucleotide sequence of SEQ ID NO:7 or SEQ ID NO:8.

In one embodiment of the invention, one or more plant cells are transformed with an expression cassette comprising the tryptophan monooxygenase nucleic acid and the indole acetamide hydrolase nucleic acid. In another embodiment of the invention, one or more plant cells are transformed with a first expression cassette comprising the tryptophan monooxygenase nucleic acid and a second expression cassette comprising the indole acetamide hydrolase nucleic acid.

In one embodiment of the invention, the tryptophan monooxygenase nucleic acid and the indole acetamide hydrolase nucleic acid are operably linked to one tissue-preferred promoter. In another embodiment, the tryptophan monooxygenase nucleic acid is operably linked to a first tissue-preferred promoter and the indole acetamide hydrolase nucleic acid is operably linked to a second tissue-preferred promoter. Preferably, the tissue-preferred promoter is a meristem-preferred promoter.

In another aspect, the invention provides an isolated expression cassette comprising an isopentenyl transferase nucleic acid, wherein the isopentenyl transferase nucleic acid is operably linked to a developmental stage-preferred promoter or a tissue-preferred promoter. Preferably, the isopentenyl transferase nucleic acid is an Agrobacterium tumefaciens T-DNA isopentenyl transferase nucleic acid. More preferably, the isopentenyl transferase nucleic acid comprises a polynucleotide sequence selected from the group consisting of: a polynucleotide of SEQ ID NO:1; a polynucleotide encoding a polypeptide of SEQ ID NO:2; a polynucleotide having at least 80% sequence identity with the nucleotide sequence of SEQ ID NO:1; a polynucleotide encoding a polypeptide having at least 80% sequence identity with the polypeptide of SEQ ID NO:2; and a polynucleotide hybridizing under stringent conditions to the nucleotide sequence of SEQ ID NO:1.

In one embodiment, the isopentenyl transferase nucleic acid is operably linked to a developmental stage-preferred promoter, wherein said developmental stage-preferred promoter is not a senescence-preferred promoter. Preferably, the developmental stage-preferred promoter is a germination-preferred promoter. More preferably, the germination-preferred promoter is an early germination-preferred promoter. In one embodiment, the germination-preferred promoter is a GA4H promoter from Arabidopsis thaliana.

In another embodiment, the isopentenyl transferase nucleic acid is operably linked to a tissue-preferred promoter. Preferably, the tissue-preferred promoter is a meristem-preferred promoter. More preferably, the tissue-preferred promoter is a root-preferred promoter. Most preferably, the root-preferred promoter is a root tip-preferred promoter. In one embodiment, the root tip-preferred promoter is a deletion derivative of a rolB promoter from Agrobacterium rhizogenes.

The invention also provides an isolated expression cassette, comprising a tryptophan monooxygenase nucleic acid operably linked to a tissue-preferred promoter. Preferably, the tryptophan monooxygenase nucleic acid is an Agrobacterium tumefaciens T-DNA tryptophan monooxygenase nucleic acid. Preferably, the tissue-preferred promoter is a meristem-preferred promoter.

The invention provides an isolated expression cassette, comprising an indole acetamide hydrolase nucleic acid operably linked to a first tissue-preferred promoter. Preferably, the indole acetamide hydrolase nucleic acid is an Agrobacterium tumefaciens T-DNA indole acetamide hydrolase nucleic acid. Preferably, the tissue-preferred promoter is a meristem-preferred promoter.

The invention further provides transgenic plant cells, plants and seeds, comprising an isolated expression cassette of the invention. The plants can be monocots or dicots. Preferably the plant is selected from the group consisting of maize, wheat, rye, oat, triticale, rice, barley, soybean, peanut, cotton, rapeseed, canola, manihot, pepper, sunflower, tagetes, solanaceous plants, potato, tobacco, eggplant, tomato, Vicia species, pea, alfalfa, coffee, cacao, tea, Salix species, oil palm, coconut, and perennial grass. Most preferably the plant is Brassica napus.

The present invention provides methods of improving plant performance, comprising a) transforming one or more plant cells with an expression cassette comprising a nucleic acid encoding an isopentenyl transferase, wherein the isopentenyl transferase nucleic acid is operably linked to a developmental stage-preferred promoter or a tissue-preferred promoter; and b) generating a transgenic plant comprising the expression cassette from the one or more plant cells; wherein said developmental-stage preferred promoter is not a senescence-preferred promoter. The present invention also provides a method of improving plant performance, comprising a) transforming one or more plant cells with one or more nucleic acids encoding either or both a tryptophan monooxygenase and an indole acetamide hydrolase, wherein the tryptophan monooxygenase nucleic acid and the indole acetamide hydrolase nucleic acid are operably linked to one or more tissue-preferred promoters and are expressed in the one or more plant cells; and b) generating from the one or more plant cells a transgenic plant comprising the tryptophan monooxygenase and/or the indole acetamide hydrolase. The present invention also provides compositions to be used in the methods such as expression vectors and expression cassettes comprising an isopentenyl transferase polynucleotide, a tryptophan monooxygenase polynucleotide, and/or an indole acetamide hydrolase polynucleotide operably linked to one or more developmental stage-preferred promoters or one or more tissue-preferred promoters.

It is to be understood that the tryptophan monooxygenase polynucleotide and the indole acetamide hydrolase polynucleotide can be expressed from a single mRNA or from separate mRNAs. Accordingly, the present invention encompasses expression vectors and expression cassettes comprising a tryptophan monooxygenase polynucleotide and an indole acetamide polynucleotide operably linked to one tissue-preferred promoter. When the tryptophan monooxygenase polynucleotide and the indole acetamide hydrolase polynucleotide are expressed from a single mRNA, the proteins can be translationally fused or can have separate translational initiation sites. When the tryptophan monooxygenase polynucleotide and the indole acetamide hydrolase polynucleotide are translationally fused, the translated protein can be a fusion protein (either protein being first) or the translated protein can be cleaved post-translationally into a tryptophan monooxygenase protein and an indole acetamide hydrolase protein. The present invention also encompasses expression vectors and expression cassettes comprising a tryptophan monooxygenase polynucleotide operably linked to a first tissue-preferred promoter and an indole hydrolase acetamide polynucleotide operably linked to a second tissue-preferred promoter.

The present invention also encompasses plants and plant cells comprising a tryptophan monooxygenase polynucleotide and the indole acetamide hydrolase polynucleotide, each operably linked to a tissue-preferred promoter, wherein the polynucleotides reside on separate expression vectors. Separate expression vectors can be co-transformed or consecutively transformed. However, in a preferred embodiment, the tryptophan monooxygenase polynucleotide and the indole acetamide hydrolase polynucleotide are on the same expression vector.

Accordingly, the present invention includes a method of increasing a plant's resistance to drought including the following steps: 1) transforming one or more plant cells with an expression cassette comprising an isopentenyl transferase polynucleotide, a tryptophan monooxygenase polynucleotide, and/or an indole acetamide hydrolase polynucleotide operably linked to a root-preferred promoter, and 2) generating from the one or more plant cells the transgenic plant. The present invention further includes a method of increasing root growth in a plant including the steps of: 1) transforming one or more plant cells with an expression cassette comprising an isopentenyl transferase polynucleotide, a tryptophan monooxygenase polynucleotide, and/or an indole acetamide hydrolase polynucleotide operably linked to a root-preferred promoter, and 2) generating from the one or more plant cells the transgenic plant. In a preferred embodiment of these two methods, the expression cassette comprises an isopentenyl transferase polynucleotide and a root tip-preferred promoter derived from a rolB promoter, preferably, a rolB promoter from Agrobacterium rhizogenes. Both the isopentenyl transferase polynucleotide and rolB promoter are described in more detail below.

Increased branching provides increased flowering and seed set, both of which are preferred in species such as Brassica napus. Accordingly, one aspect of the invention includes a method of modulating branching in a plant comprising transforming one or more plant cells with an expression cassette comprising an isopentenyl transferase polynucleotide, a tryptophan monooxygenase polynucleotide, and/or an indole acetamide hydrolase operably linked to one or more meristem-preferred promoters, and generating from the one or more plant cells the transgenic plant. In one embodiment, plant branching is increased when the expression cassette comprises an isopentenyl transferase polynucleotide operably linked to a meristem-preferred promoter. In a preferred embodiment, the plant is a Brassica species plant, and more preferably, a Brassica napus plant.

At the same time that increased branching can be preferred in species such as Brassica napus, decreased branching reduces problems with tillering encountered in maize species. Plant branching is decreased when the plant comprises a tryptophan monooxygenase polynucleotide and an indole acetamide hydrolase polynucleotide operably linked to one or more meristem-preferred promoters. In a preferred embodiment, the plant is maize, more preferably a Zea species plant, and most preferably, a Zea mays plant.

Further described herein are methods of increasing a plant seedling's vigor comprising, increasing cytokinin levels in a plant seed during germination and generating the seedling from the seed. Cytokinin levels can be increased by transforming one or more plant cells with an expression cassette comprising an isopentenyl transferase polynucleotide operably linked to a germination-preferred promoter, and producing a plant seedling from the one or more plant cells. In a preferred embodiment, the germination-preferred promoter is a GA4H promoter from Arabidopsis thaliana. As used herein, the term “vigor” refers to an active, healthy or well-balanced growth. An increase in a transgenic plant seedling's vigor is evidenced by that seedling's increased ability to develop into a mature plant as compared to a wild-type (or non-transgenic) variety of the plant seedling. Methods of measuring seedling vigor are known to those skilled in the art, and include but are not limited to measurement of shoot length, cotyledon size, root length or root mass. Preferably, seedling vigor is determined by measuring cotyledon size. Preferably, a transgenic plant seedling's vigor is increased by at least 5% in comparison to the vigor of a control, wild-type plant seedling. More preferably, a transgenic plant seedling's vigor is increased by greater than 10%, 15%, 20%, 25%, 30%, 35%, 40%, 45%, 50%, 55%, 60%, 65%, 70%, 75%, 80%, 85%, 90%, 95%, or 100% in comparison to the vigor of a control, wild-type plant seedling.

As used herein, the term “isopentenyl transferase” (EC 2.5.1.27) refers to an enzyme that converts isopentenyl diphosphate and adenosine monophosphate into isopentenyl-adenosine-5-monophosphate and pyrophosphate. Isopentenyl transferases that may be encompassed by the present invention include, but are not limited to, those having NCBI Accession Numbers AE009419, AY052773, AY052768, AF109376, AB025109, Z29635, X14410, M91610 and M15991. Preferably, the isopentenyl transferase is an Agrobacterium tumefaciens or an A. rhizogenes T-DNA isopentenyl transferase. The A. tumefaciens T-DNA isopentenyl transferase is encoded by the ipt oncogene.

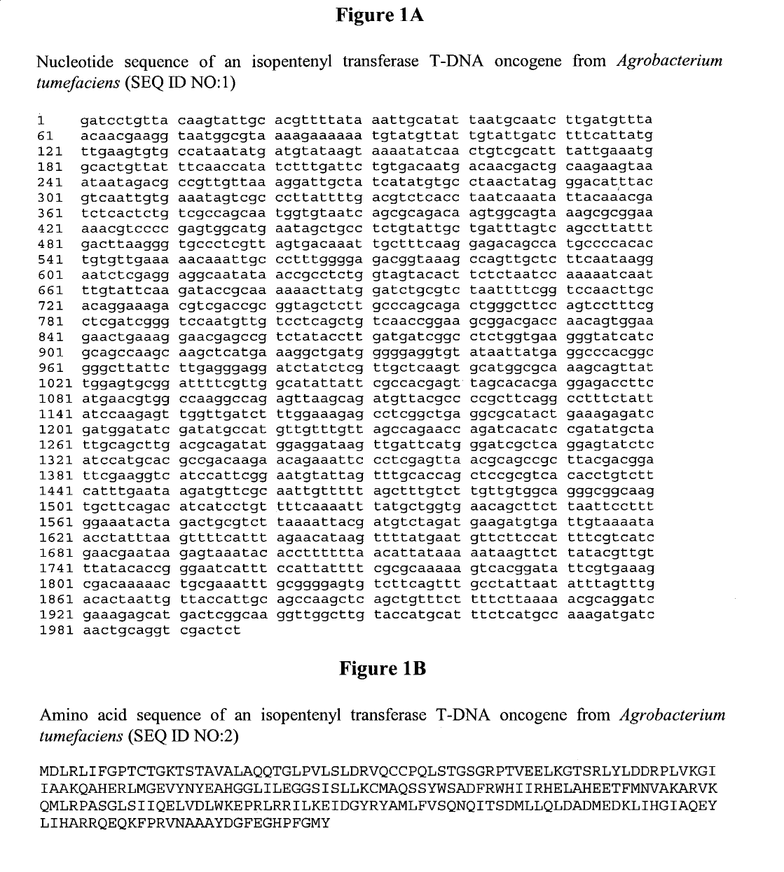

The nucleotide and amino acid sequences of an ipt oncogene from Agrobacterium tumefaciens are shown in FIG. 1 as SEQ ID NOs:1 and 2, respectively. Accordingly, the ipt oncogene of the present invention can comprise a polynucleotide sequence selected from the group consisting of 1) a polynucleotide of SEQ ID NO:1; 2) a polynucleotide encoding a polypeptide of SEQ ID NO:2; 3) a polynucleotide having at least 80% sequence identity with the nucleotide sequence of SEQ ID NO:1; 4) a polynucleotide encoding a polypeptide having at least 80% sequence identity with the polypeptide of SEQ ID NO:2; and 5) a polynucleotide hybridizing under stringent conditions to the nucleotide sequence of SEQ ID NO:1.

As also used herein, the term “tryptophan monooxygenase” (EC 1.13.12.3) refers to an enzyme that converts L-tryptophan and oxygen into indole-3-acetamide, carbon dioxide and water. Tryptophan monooxygenases that may be encompassed by the present invention include, but are not limited to, those having NCBI Accession Numbers AE007927, L33867, AF126446, AF126447, AB032122, AB025110, Z18270, M91609 and U04358. Preferably, the tryptophan monooxygenase is an Agrobacterium tumefaciens or an A. rhizogenes T-DNA tryptophan monooxygenase. The A. tumefaciens T-DNA tryptophan monooxygenase is encoded by the iaaM oncogene.

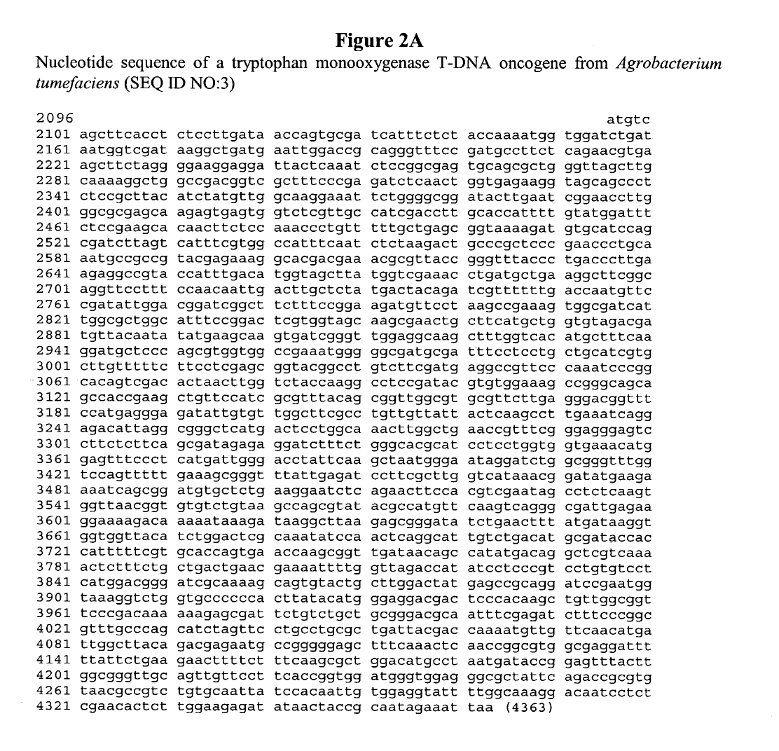

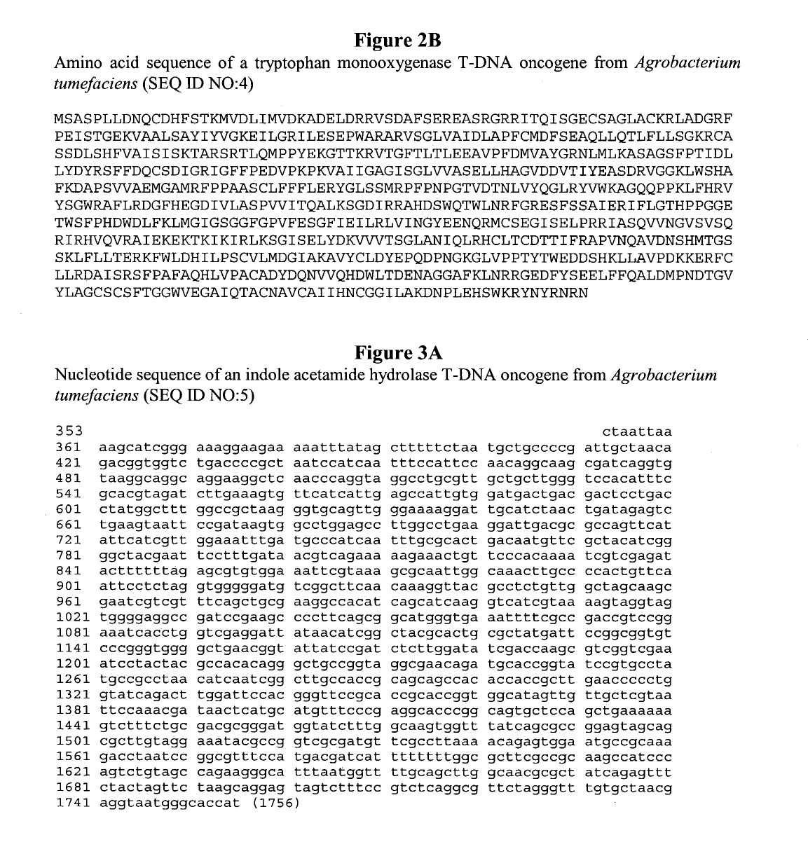

The nucleotide and amino acid sequences of an iaaM oncogene from Agrobacterium tumefaciens are shown in FIG. 2 as SEQ ID NOs:3 and 4, respectively. Accordingly, the iaaM oncogene of the present invention can comprise a polynucleotide sequence selected from the group consisting of 1) a polynucleotide of SEQ ID NO:3; 2) a polynucleotide encoding a polypeptide of SEQ ID NO:4; 3) a polynucleotide having at least 80% sequence identity with the nucleotide sequence of SEQ ID NO:3; 4) a polynucleotide encoding a polypeptide having at least 80% sequence identity with the polypeptide of SEQ ID NO:4; and 5) a polynucleotide hybridizing under stringent conditions to the nucleotide sequence of SEQ ID NO:3.

As further used herein, the term “indole acetamide hydrolase” (EC 3.5.1) refers to an enzyme that converts indole-3-acetamide (IAM) into indole-3-acetic acid (IAA). Indole acetamide hydrolases that may be encompassed by the present invention include, but are not limited to, those having NCBI Accession Numbers AE009419, AF029344, AB028643, AB025110 and M91609. Preferably, the indole acetamide hydrolase is an Agrobacterium tumefaciens or an A. rhizogenes T-DNA indole acetamide hydrolase. The A. tumefaciens T-DNA indole acetamide hydrolase is encoded by the iaaH oncogene.

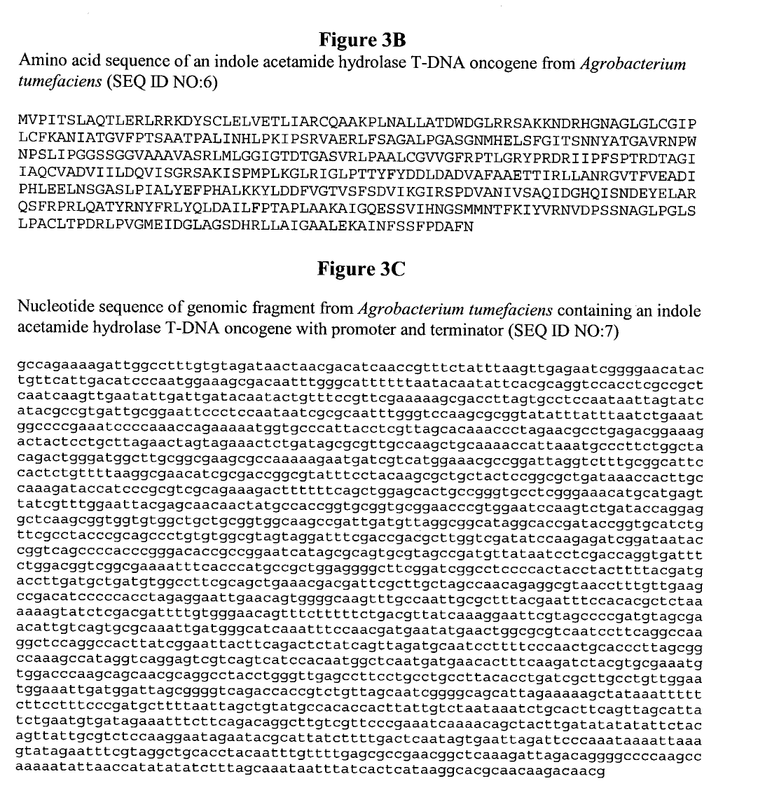

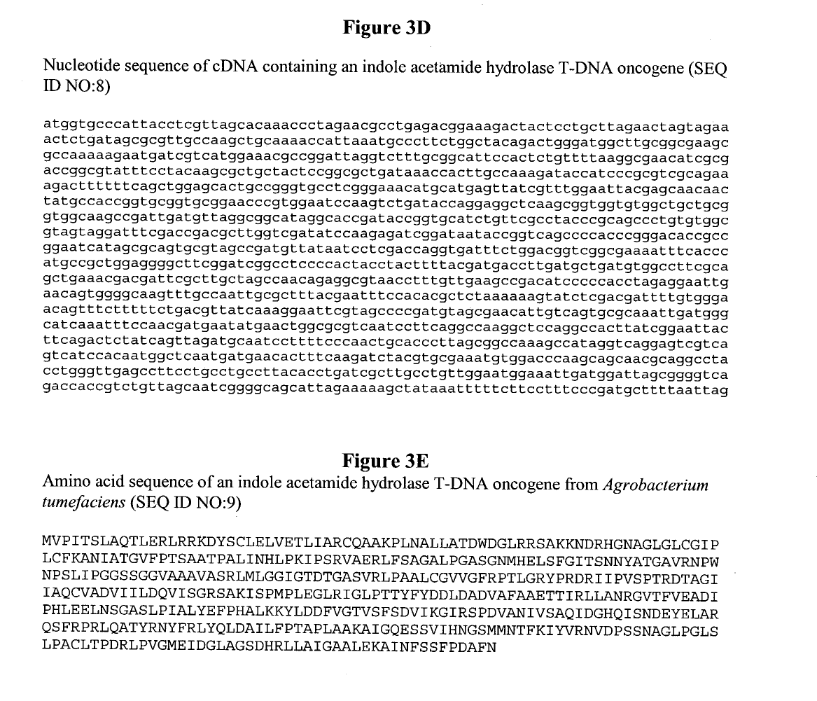

The nucleotide sequences of an iaaH oncogene from Agrobacterium tumefaciens are shown in FIGS. 3A, C, and D as SEQ ID NOs:5, 7, and 8, respectively. The amino acid sequences of an iaaH oncogene from Agrobacterium tumefaciens are shown in FIGS. 3B and D as SEQ ID NOs:6 and 9, respectively. Accordingly, the iaaH oncogene of the present invention can comprise a polynucleotide sequence selected from the group consisting of 1) a polynucleotide of SEQ ID NO:5 8; 2) a polynucleotide encoding a polypeptide of SEQ ID NO:6; 3) a polynucleotide having at least 80% sequence identity with the nucleotide sequence of SEQ ID NO:5; 4) a polynucleotide encoding a polypeptide having at least 80% sequence identity with the polypeptide of SEQ ID NO:6; and 5) a polynucleotide hybridizing under stringent conditions to the nucleotide sequence of SEQ ID NO:5. In a preferred embodiment, the iaaH oncogene of the present invention can comprise a polynucleotide sequence selected from the group consisting of 1) a polynucleotide of SEQ ID NOs:7, or 8; 2) a polynucleotide encoding a polypeptide of SEQ ID NO:9; 3) a polynucleotide having at least 80% sequence identity with the nucleotide sequence of SEQ ID NOs:7, or 8; 4) a polynucleotide encoding a polypeptide having at least 80% sequence identity with the polypeptide of SEQ ID NO:9; and 5) a polynucleotide hybridizing under stringent conditions to the nucleotide sequence of SEQ ID NOs:7 or 8.

It is to be understood that as used in the specification and in the claims, “a” or “an” can mean one or more, depending upon the context in which it is used. Thus, for example, reference to “a cell” can mean that at least one cell can be utilized. Additionally, the terms “transfer DNA” and “T-DNA” refer to the portion of the Agrobacterium tumefaciens Ti plasmid that is transferred from the bacterium into the infected host plant cell. T-DNA includes the ipt, iaaM and iaaH oncogenes.

The isopentenyl transferase polynucleotides, tryptophan monooxygenase polynucleotides and indole acetamide hydrolase polynucleotides of the present invention are expressed in a plant cell. In one embodiment, controlled expression of the isopentenyl transferase polynucleotide, tryptophan monooxygenase polynucleotide or indole acetamide hydrolase polynucleotide is achieved by operably linking the polynucleotide with a regulatory sequence. As used herein, the term “operably linked” is intended to mean that the polynucleotide is linked to the regulatory sequence(s) in a manner which allows for expression of the polynucleotide (e.g., in an in vitro transcription/translation system or in a host cell when a vector or expression cassette containing the polynucleotide is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990) or see: Gruber and Crosby, in: Methods in Plant Molecular Biology and Biotechnology, eds. Glick and Thompson, Chapter 7, 89-108, CRC Press: Boca Raton, Fla., including the references therein. Regulatory sequences include those that direct constitutive expression of a nucleotide sequence in many types of host cells and those that direct expression of the nucleotide sequence only in certain host cells or under certain conditions.

In a preferred embodiment of the present invention, an isopentenyl transferase polynucleotide, a tryptophan monooxygenase polynucleotide or an indole acetamide hydrolase polynucleotide is operably linked to a developmental stage-preferred, plant tissue-preferred, or plant organ-preferred promoter, wherein the developmental stage-preferred promoter is not a senescence-preferred promoter. The developmental stage-preferred, plant tissue-preferred, or plant organ-preferred promoter can be obtained from plants, plant viruses or bacteria that contain genes that are expressed in plants, such as Agrobacterium or Rhizobium. One of skill in the art will recognize that a developmental stage-preferred promoter may drive expression of operably linked sequences at developmental stages other than the preferred developmental stage. Thus, a developmental stage-preferred promoter is one that drives expression preferentially during the preferred developmental stage, but may also lead to some expression during other developmental stages as well. One of skill in the art will also recognize that a tissue-preferred promoter may drive expression of operably linked sequences in tissues other than the target or preferred tissue. Thus, a tissue-preferred promoter is one that drives expression preferentially in the target tissue, but may also lead to some expression in other tissues as well.

Developmental stage-preferred promoters are preferentially expressed at certain stages of development. Developmental stage-preferred promoters include germination-preferred promoters and seed-preferred promoters. The term “germination” refers to the process where a seed, spore or zygote begins to sprout, grow, or develop, usually after it has been dormant for a time while waiting for the right growing conditions. Germination-preferred promoters are known in the art and include, but are not limited to, the GA4H promoter from Arabidopsis thaliana, the cysteine proteinase promoter from carrot, legumes, Pisum sativum and chickpea, the rice carboxypeptidase promoter, the mitochondrila HSP60 promoter from maize and Arabidopsis thaliana, the alpha-amylase promoter from Vigna mungro, maize, rice and barley, gibberellic acid biosynthetic genes from rice, wheat, maize, tobacco, tomato, potato, Arabidopsis thaliana, and the Arabidopsis thaliana, EPR1 extensin-like gene. Seed-preferred promoters can also be included in the group of developmental stage-preferred promoters as these are preferentially expressed during seed development and/or germination. Examples of seed-preferred promoters include, but are not limited to, cellulose synthase (celA), Cim1, gamma-zein, globulin-1, maize 19 kD zein (cZ19B1) and the like.

In one embodiment of the present invention, the developmental stage-preferred promoter is a germination-preferred promoter, and more preferably, an early germination preferred promoter. Most preferably, the developmental stage-preferred promoter is a GA4H promoter from Arabidopsis thaliana. Expression of an isopentenyl transferase polynucleotide under the control of a germination-preferred promoter results in increased seedling vigor.

Tissue- and organ-preferred promoters include those that are preferentially expressed in certain tissues or organs, such as leaves, roots, seeds, or xylem. Examples of tissue preferred and organ preferred promoters include, but are not limited to, anther-preferred, fruit-preferred, integument-preferred, leaf-preferred, male tissue-preferred, meristem-preferred, ovule-preferred, pedicel-preferred, pericarp-preferred, petal-preferred, pollen-preferred, root-preferred, seed-preferred, sepal-preferred, silique-preferred, stalk-preferred, stem-preferred, stigma-preferred and tuber-preferred promoters. Seed preferred promoters can be embryo-preferred, endosperm preferred and seed coat-preferred (see Thompson et al., 1989 BioEssays 10:108).

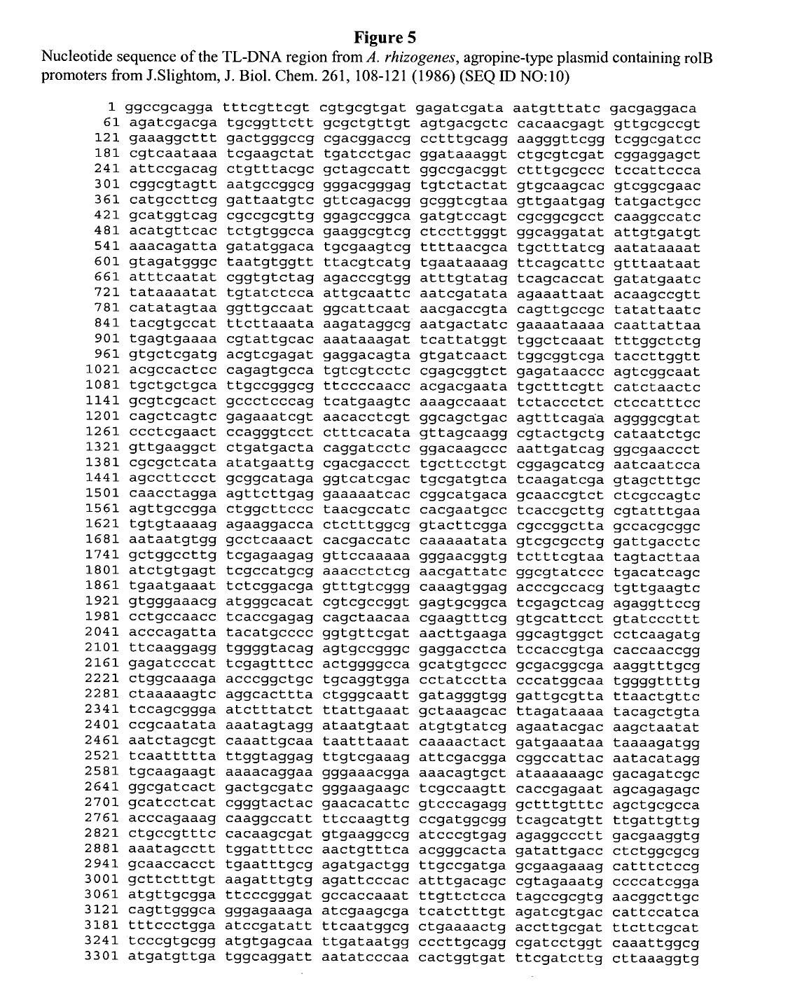

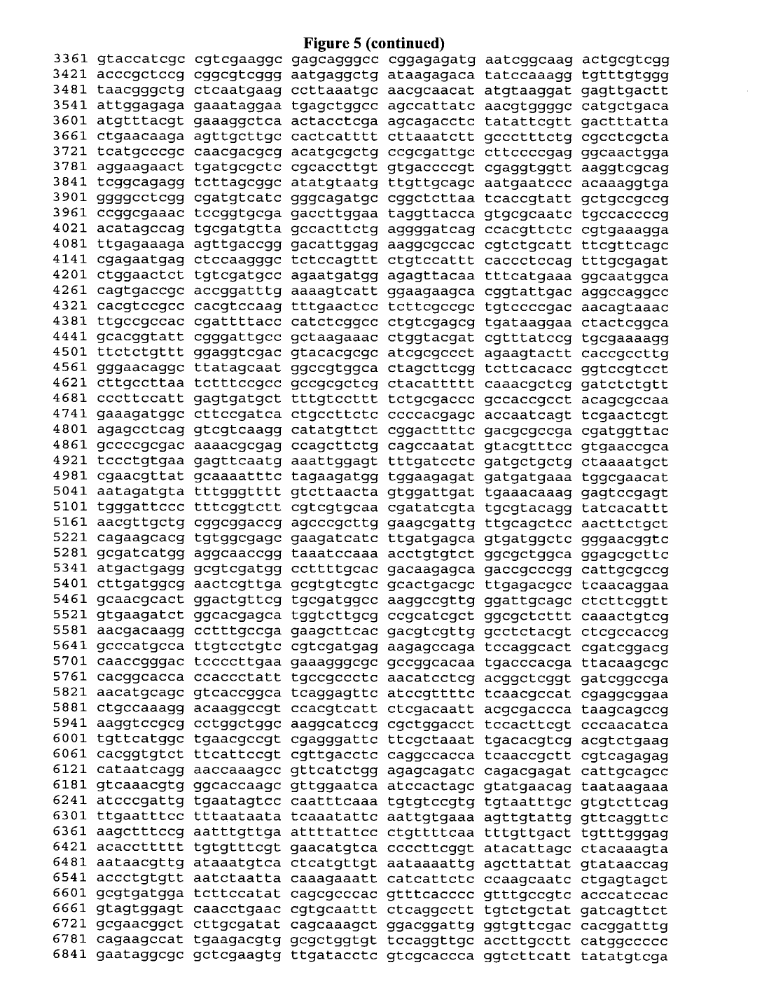

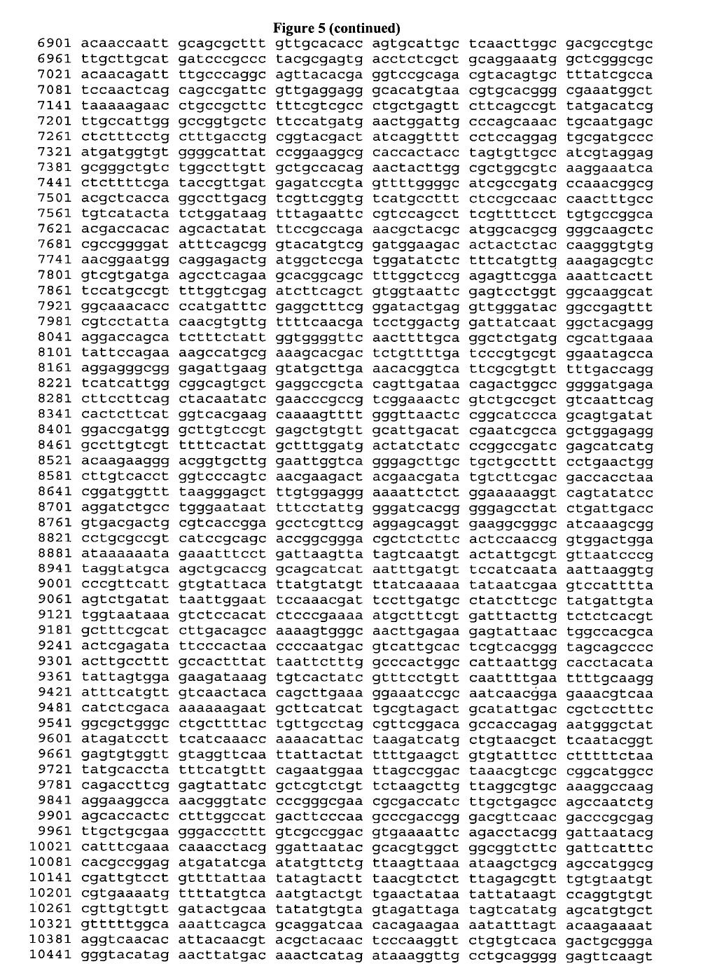

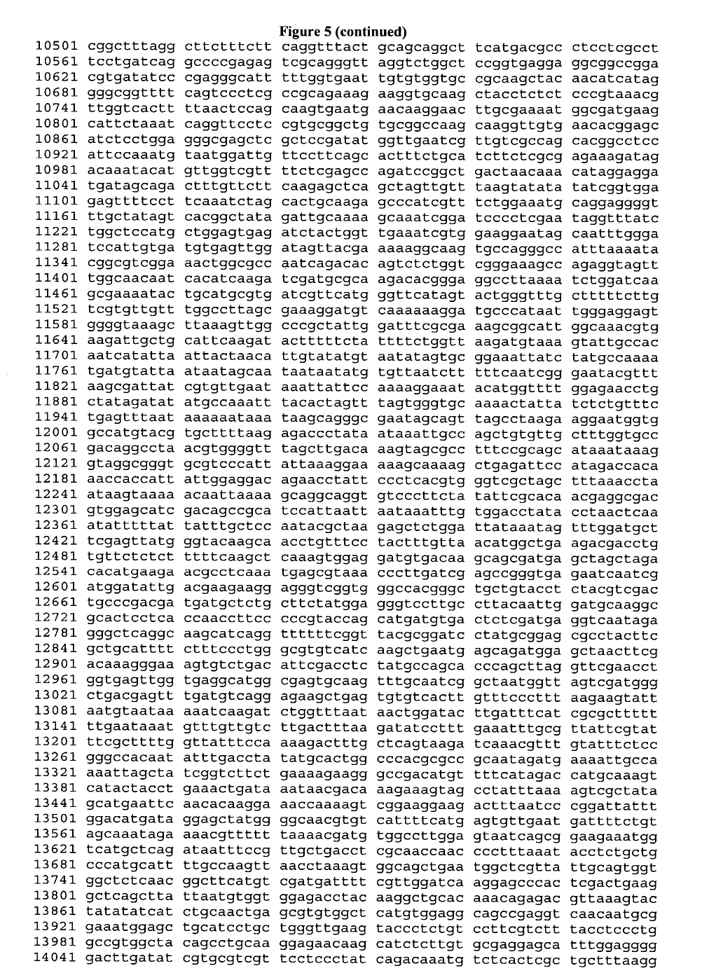

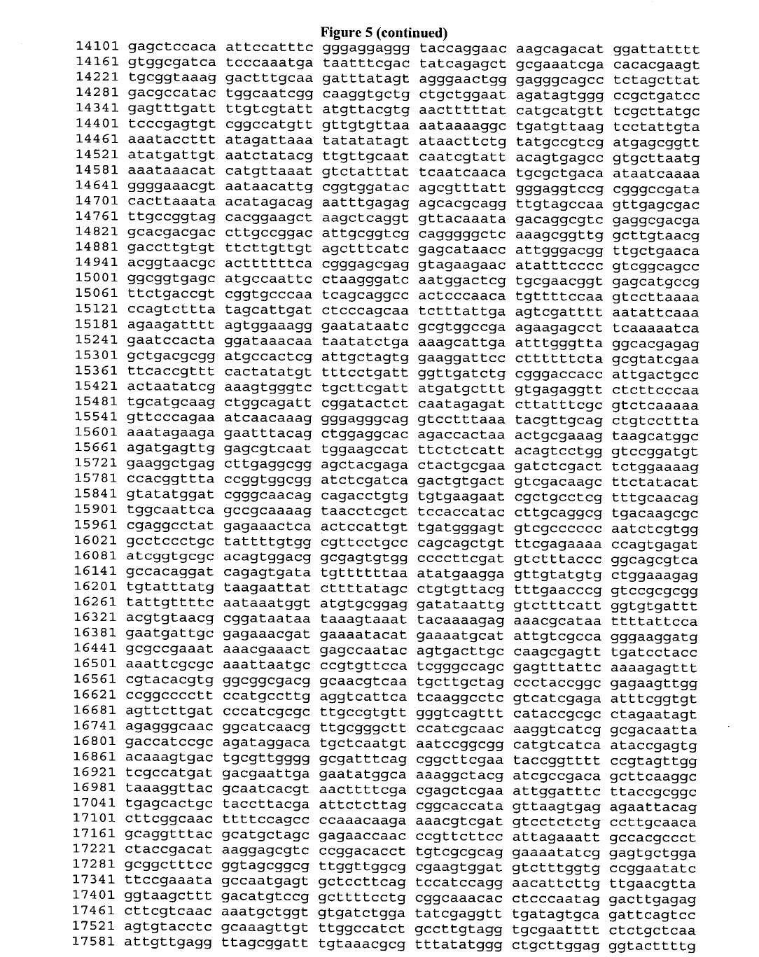

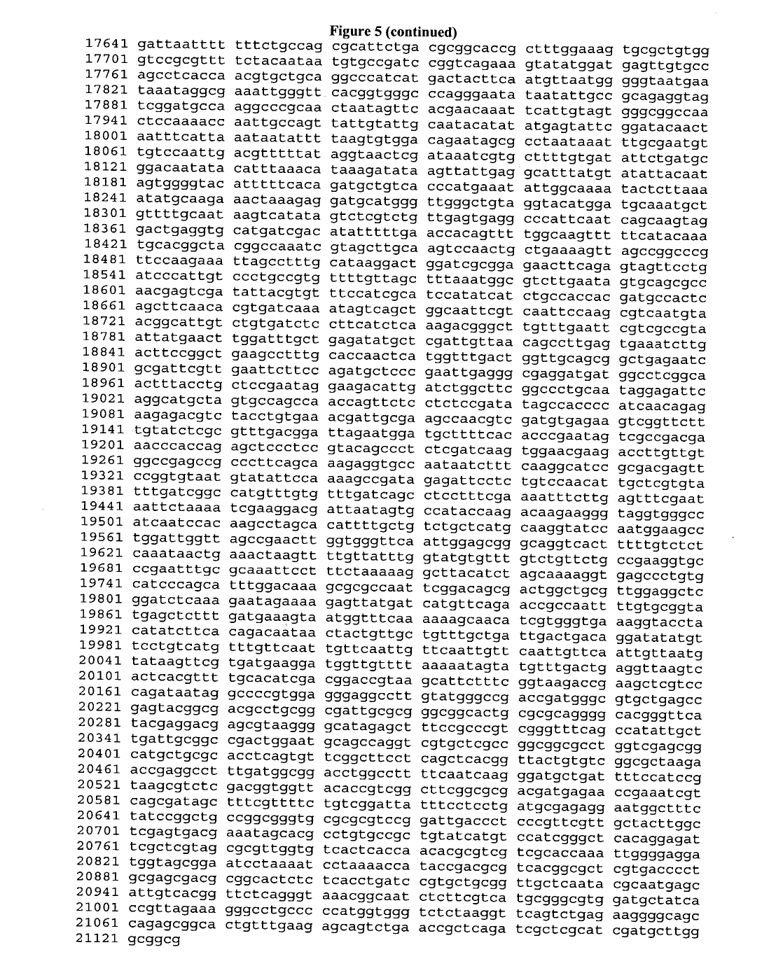

In one embodiment of the present invention, the tissue-preferred promoter is a root-preferred promoter, and more preferably, a root-tip preferred promoter. Most preferably, the root-tip preferred promoter is a deletion derivative of the Agrobacterium rhizogenes rolB promoter. Examples of deletion derivatives of the rolB promoter showing root-tip preferred expression are disclosed in Capone et al., 1994 Plant Mol. Biol. 25:681-691. Some preferred rolB deletion promoters are the B341, B341-D1 and B623 constructs shown in FIG. 2 of the aforementioned reference. FIG. 5 herein shows the nucleotide sequence of the TL-DNA region from A. rhizogenes, agropine-type plasmid containing rolB promoters as SEQ ID NO:10. From this sequence, the creation of the deletion derivates can be made according to the description in Capone et al., 1991 Plant Mol. Biol. 16:427-436.

Other examples of root-preferred promoters are known to those skilled in the art and include but are not limited to the mas and ARSK1 promoters from A. thaliana, the PtxA promoter from pea and the SbHRGP3 promoter from soybean. Transforming a plant or plant cell with an expression cassette containing a root tip-preferred promoter operably linked to an isopentenyl transferase polynucleotide results in the plant's increased resistance to drought and/or increased root growth. Increases in drought resistance and root growth are made with comparison to wild-type plants as described in more detail below. Root growth can be measured as root mass, and/or growth rate as measured on plates, or time to emerge from the bottom of a pot.

In another preferred embodiment, the tissue-preferred promoter is a meristem-preferred promoter. Meristem-preferred promoters are preferentially expressed during plant cell growth and division. Specifically, meristem is a growing region of a plant in which cells that have retained their embryonic characteristics, or reverted to them secondarily, divide to produce new cells. Non-limiting examples of meristem-preferred promoters are described in U.S. Pat. Nos. 6,329,574, 6,239,329, 6,025,545, 5,990,390 and 5,880,330. Preferably, the meristem-preferred promoter is operably linked to an isopentenyl transferase polynucleotide, a tryptophan monooxygenase polynucleotide and/or an indole acetamide hydrolase polynucleotide. Transforming a plant or plant cell with an expression cassette comprising a meristem-preferred promoter operably linked to an isopentenyl transferase polynucleotide leads to increased plant branching. Plant branching is decreased by transforming a plant with an expression cassette comprising one or more meristem-specific promoters operably linked to a tryptophan monooxygenase polynucleotide and an indole acetamide hydrolase polynucleotide. Increases and decreases in branching are made with comparison to wild-type plants as described in more detail below.

Other suitable tissue-preferred or organ-preferred promoters include the napin-gene promoter from rapeseed (U.S. Pat. No. 5,608,152), the USP-promoter from Vicia faba (Baeumlein et al., 1991 Mol Gen Genet. 225(3):459-67), the oleosin-promoter from Arabidopsis (PCT Application No. WO 98/45461), the phaseolin-promoter from Phaseolus vulgaris (U.S. Pat. No. 5,504,200), the Bce4-promoter from Brassica (PCT Application No. WO 91/13980) or the legumin B4 promoter (LeB4; Baeumlein et al., 1992 Plant Journal, 2(2):233-9) as well as promoters conferring seed specific expression in monocot plants like maize, barley, wheat, rye, rice, etc. Suitable promoters to note are the lpt2- or lpt1-gene promoter from barley (PCT Application No. WO 95/15389 and PCT Application No. WO 95/23230) or those described in PCT Application No. WO 99/16890 (promoters from the barley hordein-gene, rice glutelin gene, rice oryzin gene, rice prolamin gene, wheat gliadin gene, wheat glutelin gene, oat glutelin gene, Sorghum kasirin-gene and rye secalin gene).

In another embodiment of the present invention, controlled expression of an isopentenyl transferase polynucleotide, a tryptophan monooxygenase polynucleotide or an indole acetamide hydrolase polynucleotide is achieved or augmented by using DNA binding domains and response elements from heterologous sources (i.e., DNA binding domains from non-plant sources). An example of such a heterologous DNA binding domain is the LexA DNA binding domain (Brent and Ptashne, 1985 Cell 43:729-736). In yet another embodiment, transcription of the isopentenyl transferase polynucleotide, tryptophan monooxygenase polynucleotide or indole acetamide hydrolase polynucleotide is modulated using zinc-finger derived transcription factors (ZFPs) as described in Greisman and Pabo, 1997 Science 275:657 and manufactured by Sangamo Biosciences, Inc. These ZFPs comprise both a DNA recognition domain and a functional domain that causes activation or repression of a target nucleic acid such as an isopentenyl transferase polynucleotide, a tryptophan monooxygenase polynucleotide or an indole acetamide hydrolase polynucleotide. Therefore, activating and repressing ZFPs can be created that specifically recognize the isopentenyl transferase polynucleotide, tryptophan monooxygenase polynucleotide or indole acetamide hydrolase polynucleotide promoters described above and used to increase or decrease polynucleotide expression in a plant.

It is to be understood that expression vectors of the present invention can comprise additional genes of interest, operably linked to a promoter expressible in a plant. Such promoters can be constitutive, inducible, developmental stage-preferred, plant tissue-preferred, or plant organ-preferred. Examples of constitutive promoters include the CaMV 19S and 35S promoters (Odell et al., 1985 Nature 313:810-812), the sX CaMV 35S promoter (Kay et al., 1987 Science 236:1299-1302) the Sep1 promoter, the rice actin promoter (McElroy et al., 1990 Plant Cell 2:163-171), the Arabidopsis actin promoter, the ubiquitin promoter (Christensen et al., 1989 Plant Molec. Biol. 18:675-689); pEmu (Last et al., 1991 Theor Appl Genet 81:581-588), the figwort mosaic virus 35S promoter, the Smas promoter (Velten et al., 1984 EMBO J. 3:2723-2730), the GRP1-8 promoter, the cinnamyl alcohol dehydrogenase promoter (U.S. Pat. No. 5,683,439), promoters from the T-DNA of Agrobacterium, such as mannopine synthase, nopaline synthase, and octopine synthase, the small subunit of ribulose biphosphate carboxylase (ssuRUBISCO) prompter, and the like. These constitutive promoters can be operably linked to additional genes of interest such as an AHAS gene (acetohydroxyacid synthase gene) or an oil trait gene. In a preferred embodiment, the AHAS is mutated such that in confers imidazolinone resistance to the plant in which it is expressed. Examples of such constructs are described in Example 1.

Inducible promoters are active under certain environmental conditions, such as the presence or absence of a nutrient or metabolite, heat or cold, light, pathogen attack, anaerobic conditions, and the like. For example, the hsp80 promoter from Brassica is induced by heat shock, the PPDK promoter is induced by light, the PR-1 promoter from tobacco, Arabidopsis and maize are inducible by infection with a pathogen, and the Adh1 promoter is induced by hypoxia and cold stress. Chemically inducible promoters are especially suitable when time specific gene expression is desired. Examples of chemically inducible promoters are a salicylic acid inducible promoter (PCT Application No. WO 95/19443), a tetracycline inducible promoter (Gatz et al., 1992 Plant J. 2:397-404) and an ethanol inducible promoter (PCT Application No. WO 93/21334).

Other examples of inducible promoters are stress-inducible promoters. Stress inducible promoters include, but are not limited to, Cor78 (Chak et al., 2000 Planta 210:875-883; Hovath et al., 1993 Plant Physiol. 103:1047-1053), Cor15a (Artus et al., 1996 PNAS 93(23):13404-09), Rci2A (Medina et al., 2001 Plant Physiol. 125:1655-66; Nylander et al., 2001 Plant Mol. Biol. 45:341-52; Navarre and Goffeau, 2000 EMBO J. 19:2515-24; Capel et al., 1997 Plant Physiol. 115:569-76), Rd22 (Xiong et al., 2001 Plant Cell 13:2063-83; Abe et al., 1997 Plant Cell 9:1859-68; Iwasaki et al., 1995 Mol. Gen. Genet. 247:391-8), cDet6 (Lang and Palve, 1992 Plant Mol. Biol. 20:951-62), ADH1 (Hoeren et al., 1998 Genetics 149:479-90), KAT1 (Nakamura et al., 1995 Plant Physiol. 109:371-4), KST1 (Müller-Röber et al., 1995 EMBO 14:2409-16), Rha1 (Terryn et al., 1993 Plant Cell 5:1761-9; Terryn et al., 1992 FEBS Lett. 299(3):287-90), ARSK1 (Atkinson et al., 1997 L22302 (GenBank Accession #) and PCT Application No. WO 97/20057), PtxA (Plesch et al., X67427 (GenBank Accession #), SbHRGP3 (Ahn et al., 1996 Plant Cell 8:1477-90), GH3 (Liu et al., 1994 Plant Cell 6:645-57), the pathogen inducible PRP1-gene promoter (Ward et al., 1993 Plant. Mol. Biol. 22:361-366), the heat inducible hsp80-promoter from tomato (U.S. Pat. No. 5,187,267), cold inducible alpha-amylase promoter from potato (PCT Application No. WO 96/12814) or the wound-inducible pinII-promoter (European Patent No. 375091). For other examples of drought, cold, and salt-inducible promoters, such as the RD29A promoter, see Yamaguchi-Shinozalei et al., (1993 Mol. Gen. Genet. 236:331-340).

Additional promoters useful in the expression vectors of the invention include, but are not limited to, the major chlorophyll a/b binding protein promoter, histone promoters, the Ap3 promoter, the β-conglycin promoter, the napin promoter, the soy bean lectin promoter, the maize 15 kD zein promoter, the 22 kD zein promoter, the 27 kD zein promoter, the g-zein promoter, the waxy, shrunken 1, shrunken 2 and bronze promoters, the Zm13 promoter (U.S. Pat. No. 5,086,169), the maize polygalacturonase promoters (PG) (U.S. Pat. Nos. 5,412,085 and 5,545,546) and the SGB6 promoter (U.S. Pat. No. 5,470,359), as well as synthetic or other natural promoters.

The invention further provides transgenic plant cells, transgenic plant parts and transgenic plants containing the isopentenyl transferase polynucleotides, tryptophan monooxygenase polynucleotides and/or the indole acetamide hydrolase polynucleotides described herein. As used herein, the term “transgenic” refers to any plant, plant cell, callus, plant tissue or plant part, that contains all or part of at least one recombinant polynucleotide. In many cases, all or part of the recombinant polynucleotide is stably integrated into a chromosome or stable extra-chromosomal element, so that it is passed on to successive generations. The plant cell includes, but is not limited to, a protoplast, gamete producing cell, and a cell that regenerates into a whole plant. Plant parts include, but are not limited to, stems, roots, ovules, stamens, leaves, embryos, meristematic regions, callus tissue, gametophytes, sporophytes, pollen, microspores and the like. The transgenic plants of the present invention can be monocotyledonous (monocots) or dicotyledonous (dicots). The transgenic plant can be selected from the group consisting of, but not limited to, crop plants such as barley, canola, cotton, maize, manihot, oat, peanut, pepper, rape, rapeseed, or rapeseed oil, rice, rye, soybean, sunflower, tagetes, triticale and wheat; solanaceous plants like potato, tobacco, eggplant, and tomato; Vicia species; pea; alfalfa; bushy plants such as coffee, cacao, tea; Salix species; trees such as oil palm and coconut; perennial grasses and forage crops. Forage crops include, but are not limited to, Wheatgrass, Canarygrass, Bromegrass, Wildrye Grass, Bluegrass, Orchardgrass, Alfalfa, Salfoin, Birdsfoot Trefoil, Alsike Clover, Red Clover and Sweet Clover. In one embodiment, the transgenic plant is male sterile. In a preferred embodiment, the plant is a Brassica species plant, and more preferably, a Brassica napus plant. In another preferred embodiment, the plant is a Zea species plant, and more preferably, a Zea mays plant.

Also provided is a plant seed produced by a transgenic plant transformed by an isopentenyl transferase polynucleotide, a tryptophan monooxygenase polynucleotide or an indole acetamide hydrolase polynucleotide, or an expression vector or cassette comprising one or more of the aforementioned polynucleotides, wherein the seed contains the polynucleotide, and wherein the plant is true breeding for an improved plant characteristic described herein as compared to a wild-type variety of the plant. Improved plant characteristics include increased root growth, increased drought resistance, increased seedling vigor and increased or decreased branching. The invention further provides a seed produced by a transgenic plant expressing an isopentenyl transferase polynucleotide, a tryptophan monooxygenase polynucleotide and/or an indole acetamide hydrolase polynucleotide, wherein the seed contains the isopentenyl transferase polynucleotide, tryptophan monooxygenase polynucleotide and/or the indole acetamide hydrolase polynucleotide, and wherein the plant is true breeding for an improved plant characteristic described herein as compared to a wild type variety of the plant. The invention also provides an agricultural product produced by any of the above- or below-described transgenic plants, plant parts and plant seeds. Agricultural products include, but are not limited to, plant extracts, proteins, amino acids, carbohydrates, fats, oils, polymers, vitamins, and the like.

As used herein, the term “variety” refers to a group of plants within a species that share constant characteristics that separate them from the typical form and from other possible varieties within that species. While possessing at least one distinctive trait, a variety is also characterized by some variation between individuals within the variety, based primarily on the Mendelian segregation of traits among the progeny of succeeding generations. A variety is considered “true breeding” for a particular trait if it is genetically homozygous for that trait to the extent that, when the true-breeding variety is self-pollinated, a significant amount of independent segregation of the trait among the progeny is not observed. In the present invention, the trait arises from the transgenic expression of one or more DNA sequences introduced into a plant variety.

The isopentenyl transferase polynucleotides, tryptophan monooxygenase polynucleotides and indole acetamide hydrolase polynucleotides with which the plants, plant parts and plant cells are transformed are preferably recombinant polynucleotide sequences. However, it to be understood that the polynucleotides of the invention may be produced by any means, including genomic preparations, cDNA preparations, in vitro synthesis, RT-PCR and in vitro or in vivo transcription. For the purposes of the invention, the term “recombinant polynucleotide” refers to a polynucleotide that has been altered, rearranged or modified by genetic engineering. Examples include any cloned polynucleotide, and polynucleotides that are linked or joined to heterologous sequences. The term “recombinant” does not refer to alterations to polynucleotides that result from naturally occurring events, such as spontaneous mutations.

The terms “polynucleotide” and “nucleic acid” are used interchangeably herein and refer to RNA or DNA that is linear or branched, single or double stranded, or a hybrid thereof such as a RNA/DNA hybrid. These terms also encompass coding regions such as the polynucleotide sequences shown in SEQ ID NO:1, SEQ ID NO:3, SEQ ID NO:5, SEQ ID NO:7, and SEQ ID NO:8, and optionally, non-coding sequences. Non-coding sequence is untranslated sequence located at both the 3′ and 5′ ends of the coding region of the gene. Moreover, the isopentenyl transferase polynucleotides, tryptophan monooxygenase polynucleotides and indole acetamide hydrolase polynucleotides of the invention can comprise a portion of the coding region of one of the sequences in SEQ ID NO:1, SEQ ID NO:3, SEQ ID NO:5, SEQ ID NO:7, and SEQ ID NO:8, for example, a fragment encoding a biologically active portion of an isopentenyl transferase polypeptide, a tryptophan monooxygenase polypeptide or an indole acetamide hydrolase polypeptide.

As used herein, the term “polypeptide” refers to a chain of at least four amino acids joined by peptide bonds. The chain may be linear, branched, circular or combinations thereof. In a preferred embodiment, the isopentenyl transferase polypeptide comprises a sequence shown in SEQ ID NO:2, the tryptophan monooxygenase polypeptide comprises a sequence shown in SEQ ID NO:4 and the indole acetamide hydrolase polypeptide comprises a sequence shown in SEQ ID NO:6 or SEQ ID NO:9. Preferably, the isopentenyl transferase polypeptide is encoded by an ipt oncogene, the tryptophan monooxygenase polypeptide is encoded by an iaaM oncogene and the indole acetamide hydrolase polypeptide is encoded by an iaaH oncogene. More preferably, the isopentenyl transferase polypeptide is encoded by a polynucleotide sequence comprising SEQ ID NO:1, the tryptophan monooxygenase polypeptide is encoded by a polynucleotide sequence comprising SEQ ID NO:3 and the indole acetamide hydrolase polypeptide is encoded by a polynucleotide sequence comprising SEQ ID NOs:7 or 8.

It is to be understood that the isopentenyl transferase, tryptophan monooxygenase and indole acetamide hydrolase polypeptides of the present invention include those encoded by a polynucleotide sequence selected from the group consisting of SEQ ID NO:1, SEQ ID NO:3, SEQ ID NO:5, SEQ ID NO:7, and SEQ ID NO:8 and having post-translational modifications. Post-translational modifications include in vivo and in vitro chemical derivatization of polypeptides, e.g., acetylation, carboxylation, phosphorylation, or glycosylation, and such modifications may occur during polypeptide synthesis or processing or following treatment with isolated modifying enzymes. In preferred embodiments, the isopentenyl transferase, tryptophan monooxygenase and indole acetamide hydrolase polypeptides of the present invention have at least 10% of the activity of a wild-type Agrobacterium tumifaciens isopentenyl transferase, tryptophan monooxygenase or indole acetamide hydrolase polypeptide. In other preferred embodiments, the isopentenyl transferase, tryptophan monooxygenase and indole acetamide hydrolase polypeptides of the present invention have at least 15%, 20%, 25%, 30%, 35%, 40%, 45%, 50%, 55%, 60%, 65%, 70%, 75%, 80%, 85%, 90%, or 95% or more of the activity of a wild-type Agrobacterium tumifaciens isopentenyl transferase, tryptophan monooxygenase or indole acetamide hydrolase polypeptide.

As used herein, the term “biologically active portion of” an isopentenyl transferase polypeptide is intended to include a portion, e.g., a domain/motif, of an isopentenyl transferase that catalyzes the conversion of isopentenyl diphosphate and adenosine monophosphate to isopentenyl-adenosine-5-monophosphate and diphosphate. A biologically active portion of a tryptophan monooxygenase polypeptide is intended to include a portion, e.g., a domain/motif, of a tryptophan monooxygenase polypeptide that catalyzes the conversion of L-tryptophan and oxygen to indole-3-acetamide (IAM). Additionally, a biologically active portion of an indole acetamide hydrolase polypeptide is intended to include a portion, e.g., a domain/motif, of an indole acetamide hydrolase polypeptide that catalyzes the conversion of indole-3-acetamide to indole-3-acetic acid (IAA). For the purposes of the present invention, the biologically active portion should have at least 10% of the activity of the corresponding wild type enzyme. Typically, biologically active portions are 5, 10, 15, 20, 30, 35, 36, 37, 38, 39, 40, 50, 100 or more amino acids in length.

The isopentenyl transferase, tryptophan monooxygenase and indole acetamide hydrolase polynucleotides of the present invention can also be chimeric or fusion polynucleotides. As used herein, a “chimeric polynucleotide” or “fusion polynucleotide” comprises an isopentenyl transferase, tryptophan monooxygenase or indole acetamide hydrolase polynucleotide operably linked to a non-isopentenyl transferase, a non-tryptophan monooxygenase or a non-indole acetamide hydrolase polynucleotide, respectively. A non-isopentenyl transferase, a non-tryptophan monooxygenase or a non-indole acetamide hydrolase polynucleotide has both a different polynucleotide sequence and encodes a protein having a different function than an isopentenyl transferase, a tryptophan monooxygenase or an indole acetamide hydrolase, respectively. Within the fusion polynucleotide, the term “operably linked” is intended to indicate that the isopentenyl transferase, tryptophan monooxygenase or indole acetamide hydrolase polynucleotide and the non-isopentenyl transferase, non-tryptophan monooxygenase or non-indole acetamide hydrolase polynucleotide, respectively, are fused to each other so that both sequences fulfill the proposed function attributed to the sequence used. The non-isopentenyl transferase, non-tryptophan monooxygenase or non-indole acetamide hydrolase polynucleotide can be fused to the N-terminus or C-terminus of the isopentenyl transferase, tryptophan monooxygenase or indole acetamide hydrolase polynucleotide, respectively.

In addition to expression vectors, plants and plant cells containing fragments and fusion polynucleotides of isopentenyl transferase, tryptophan monooxygenase and indole acetamide hydrolase polynucleotides, the present invention encompasses expression vectors, plants and plant cells containing homologs of the isopentenyl transferase, tryptophan monooxygenase and indole acetamide hydrolase polynucleotides described above. Included in the invention are homologs of an ipt T-DNA oncogene as shown in SEQ ID NOs:1 and 2, an iaaM T-DNA oncogene as shown in SEQ ID NOs:3 and 4 and an iaaH T-DNA oncogene as shown in SEQ ID NOs:5, 6, 7, 8, 9. More preferably, the iaaH T-DNA oncogene is shown in SEQ ID NOs:7, 8, and 9.

“Homologs” are defined herein as two nucleic acids or polypeptides that have similar, or “identical”, nucleotide or amino acid sequences, respectively. Homologs include allelic variants, orthologs, paralogs, agonists and antagonists of isopentenyl transferase, tryptophan monooxygenase and indole acetamide hydrolase polynucleotides and polypeptides as defined hereafter. The term “homolog” further encompasses nucleic acid molecules that differ from the nucleotide sequences shown in SEQ ID NO:1, SEQ ID NO:3, SEQ ID NO:5, SEQ ID NO:7, or SEQ ID NO:8 (and portions thereof) due to the degeneracy of the genetic code and thus encode the same polypeptide as that encoded by the nucleotide sequences shown in SEQ ID NO:1, SEQ ID NO:3, SEQ ID NO:5, SEQ ID NO:7, or SEQ ID NO:8. As used herein a “naturally occurring” polypeptide refers to a polypeptide sequence that occurs in nature. Preferably, a naturally occurring isopentenyl transferase, tryptophan monooxygenase or indole acetamide hydrolase polypeptide comprises an amino acid sequence of SEQ ID NO:2, SEQ ID NO:4, or SEQ ID NOs:6 and 9, respectively.

Nucleic acid molecules corresponding to natural allelic variants and analogs, orthologs and paralogs of an isopentenyl transferase, a tryptophan monooxygenase or an indole acetamide hydrolase can be isolated based on their identity to the nucleic acids described herein using isopentenyl transferase, tryptophan monooxygenase and indole acetamide hydrolase cDNAs, or a portion thereof, as a hybridization probe according to standard hybridization techniques under stringent hybridization conditions. In an alternative embodiment, homologs of the isopentenyl transferase, tryptophan monooxygenase or indole acetamide hydrolase polynucleotides can be identified by screening combinatorial libraries of mutants, e.g., truncation mutants, of the polynucleotides for agonist or antagonist activity. In one embodiment, a variegated library of isopentenyl transferase, tryptophan monooxygenase or indole acetamide hydrolase polynucleotide variants is generated by combinatorial mutagenesis at the nucleic acid level and is encoded by a variegated gene library. Such a variegated library can be produced by, for example, enzymatically ligating a mixture of synthetic oligonucleotides into gene sequences such that a degenerate set of potential isopentenyl transferase, tryptophan monooxygenase or indole acetamide hydrolase polynucleotide sequences is expressible as individual polypeptides, or alternatively, as a set of larger fusion polypeptides (e.g., for phage display). There are a variety of methods that can be used to produce libraries of potential homologs from a degenerate oligonucleotide sequence. Chemical synthesis of a degenerate gene sequence can be performed in an automatic DNA synthesizer, and the synthetic gene is then ligated into an appropriate expression vector. Use of a degenerate set of genes allows for the provision, in one mixture, of all of the sequences encoding the desired set of potential homolog sequences. Methods for synthesizing degenerate oligonucleotides are known in the art (see, e.g., Narang, S. A., 1983 Tetrahedron 39:3; Itakura et al., 1984 Annu. Rev. Biochem. 53:323; Itakura et al., 1984 Science 198:1056; Ike et al., 1983 Nucleic Acid Res. 11:477).

In addition, libraries of fragments of the isopentenyl transferase, tryptophan monooxygenase or indole acetamide hydrolase polynucleotide coding regions can be used to generate a variegated population of fragments for screening and subsequent selection of homologs of isopentenyl transferase, tryptophan monooxygenase or indole acetamide hydrolase polynucleotides. In one embodiment, a library of coding sequence fragments can be generated by treating a double stranded PCR fragment of an isopentenyl transferase, tryptophan monooxygenase or indole acetamide hydrolase polynucleotide coding sequence with a nuclease under conditions wherein nicking occurs only about once per molecule, denaturing the double stranded DNA, renaturing the DNA to form double stranded DNA, which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the isopentenyl transferase, tryptophan monooxygenase or indole acetamide hydrolase polynucleotides.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of isopentenyl transferase, tryptophan monooxygenase or indole acetamide hydrolase homologs. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify homologs (Arkin and Yourvan, 1992 PNAS 89:7811-7815; Delgrave et al., 1993 Polypeptide Engineering 6(3):327-331). In another embodiment, cell based assays can be exploited to analyze a variegated library, using methods well known in the art.

The present invention further provides a method of identifying a novel isopentenyl transferase, tryptophan monooxygenase or indole acetamide hydrolase polynucleotide, comprising (a) raising a specific antibody response to an isopentenyl transferase, tryptophan monooxygenase or indole acetamide hydrolase polypeptide, respectively, or a fragment thereof, as described herein; (b) screening putative isopentenyl transferase, tryptophan monooxygenase or indole acetamide hydrolase polypeptide material with the antibody, wherein specific binding of the antibody to the material indicates the presence of a potentially novel isopentenyl transferase, tryptophan monooxygenase or indole acetamide hydrolase polypeptide, respectively; and (c) analyzing the bound material in comparison to a known isopentenyl transferase, tryptophan monooxygenase or indole acetamide hydrolase polypeptide, respectively, to determine its novelty. The phrases “selectively binds” and “specifically binds” with the polypeptide refer to a binding reaction that is determinative of the presence of the polypeptide in a heterogeneous population of polypeptides and other biologics. Thus, under designated immunoassay conditions, the specified antibodies bound to a particular polypeptide do not bind in a significant amount to other polypeptides present in the sample. Selective binding of an antibody under such conditions may require an antibody that is selected for its specificity for a particular polypeptide. A variety of immunoassay formats may be used to select antibodies that selectively bind with a particular polypeptide. For example, solid-phase ELISA immunoassays are routinely used to select antibodies selectively immunoreactive with a polypeptide. See Harlow and Lane “Antibodies, A Laboratory Manual” Cold Spring Harbor Publications, New York, (1988), for a description of immunoassay formats and conditions that could be used to determine selective binding.

As stated above, the present invention includes expression vectors, plants and plant cells comprising isopentenyl transferase, tryptophan monooxygenase and indole acetamide hydrolase polynucleotides and homologs thereof. To determine the percent sequence identity of two amino acid sequences (e.g., one of the sequences of SEQ ID NO:2, SEQ ID NO:4, SEQ ID NO:6, and SEQ ID NO:9, and a homolog thereof), the sequences are aligned for optimal comparison purposes (e.g., gaps can be introduced in the sequence of one polypeptide for optimal alignment with the other polypeptide or nucleic acid). The amino acid residues at corresponding amino acid positions are then compared. When a position in one sequence (e.g., one of the sequences of SEQ ID NO:2, SEQ ID NO:4, SEQ ID NO:6, and SEQ ID NO:9) is occupied by the same amino acid residue as the corresponding position in the other sequence (e.g., a homolog of the sequence selected from the polypeptide of SEQ ID NO:2, SEQ ID NO:4, SEQ ID NO:6, and SEQ ID NO:9), then the molecules are identical at that position. The same type of comparison can be made between two nucleic acid sequences.

The percent sequence identity between the two sequences is a function of the number of identical positions shared by the sequences (i.e., percent sequence identity=numbers of identical positions/total numbers of positions×100). Preferably, the homologs included in the present invention are at least about 50-60%, preferably at least about 60-70%, and more preferably at least about 70-75%, 75-80%, 80-85%, 85-90% or 90-95%, and most preferably at least about 96%, 97%, 98%, 99% or more identical to an entire amino acid sequence shown in SEQ ID NO:2, SEQ ID NO:4, SEQ ID NO:6, or SEQ ID NO:9. In yet another embodiment, the homologs included in the present invention are at least about 50-60%, preferably at least about 60-70%, and more preferably at least about 70-75%, 75-80%, 80-85%, 85-90% or 90-95%, and most preferably at least about 96%, 97%, 98%, 99% or more identical to an entire amino acid sequence encoded by a nucleic acid sequence shown in SEQ ID NO:1, SEQ ID NO:3, SEQ ID NO:5, SEQ ID NO:7 or SEQ ID NO:8. In other embodiments, the homologs have sequence identity over at least 15 contiguous amino acid residues, more preferably at least 25 contiguous amino acid residues, and most preferably at least 35 contiguous amino acid residues of SEQ ID NO:2, SEQ ID NO:4, SEQ ID NO:6, or SEQ ID NO:9. In a further preferred embodiment, the polypeptide homolog catalyzes an enzymatic reaction as catalyzed by an isopentenyl transferase, a tryptophan monooxygenase or an indole acetamide hydrolase polypeptide as described above.

In another preferred embodiment, a polynucleotide homolog of the invention comprises a nucleotide sequence which is at least about 50-60%, preferably at least about 60-70%, more preferably at least about 70-75%, 75-80%, 80-85%, 85-90% or 90-95%, and even more preferably at least about 95%, 96%, 97%, 98%, 99% or more identical to a nucleotide sequence shown in SEQ ID NO:1, SEQ ID NO:3, SEQ ID NO:5, SEQ ID NO:7 or SEQ ID NO:8, or to a portion comprising at least 60 consecutive nucleotides thereof. The preferable length of sequence comparison for nucleic acids is at least 75 nucleotides, more preferably at least 100 nucleotides and most preferably the entire length of the coding region. In a further preferred embodiment, the polynucleotide homolog increases drought resistance or root growth, increases seedling vigor or modulates branching when controllably expressed in a plant.

For the purposes of the invention, the percent sequence identity between two polynucleotide or polypeptide sequences is determined using the Vector NTI 6.0 (PC) software package (InforMax, 7600 Wisconsin Ave., Bethesda, Md. 20814). A gap opening penalty of 15 and a gap extension penalty of 6.66 are used for determining the percent identity of two polynucleotides. A gap opening penalty of 10 and a gap extension penalty of 0.1 are used for determining the percent identity of two polypeptides. All other parameters are set at the default settings. It is to be understood that for the purposes of determining sequence identity when comparing a DNA sequence to an RNA sequence, a thymidine nucleotide is equivalent to a uracil nucleotide.

In another aspect, the invention provides an expression vector, plant or plant cell comprising a polynucleotide that hybridizes to the polynucleotide of SEQ ID NO:1, SEQ ID NO:3, SEQ ID NO:5, SEQ ID NO:7 or SEQ ID NO:8 under stringent conditions, wherein the hybridizing sequence is operably linked to a regulatable promoter. More particularly, a hybridizing sequence is at least 15 nucleotides in length and hybridizes under stringent conditions to the nucleic acid molecule comprising a nucleotide sequence of SEQ ID NO:1, SEQ ID NO:3, SEQ ID NO:5, SEQ ID NO:7 or SEQ ID NO:8. In other embodiments, the nucleic acid is at least 30, 50, 100, 250 or more nucleotides in length. In a preferred embodiment, expression of the hybridizing sequence in a plant increases cytokinin or auxin levels in the plant. In a further preferred embodiment, expression of the hybridizing sequence in a plant increases a plant's tolerance to drought, increases a plant's root growth, increases a plant seedling's vigor or modulates a plant's branching.

As used herein with regard to hybridization for DNA to DNA blot, the term “stringent conditions” refers to hybridization overnight at 60° C. in 10× Denhart's solution, 6× SSC, 0.5% SDS and 100 μg/ml denatured salmon sperm DNA. Blots are washed sequentially at 62° C. for 30 minutes each time in 3× SSC/0.1% SDS, followed by 1× SSC/0.1% SDS and finally 0.1× SSC/0.1% SDS. As also used herein, “highly stringent conditions” refers to hybridization overnight at 65° C. in 10× Denhart's solution, 6× SSC, 0.5% SDS and 100 μg/m l denatured salmon sperm DNA. Blots are washed sequentially at 65° C. for 30 minutes each time in 3× SSC/0.1% SDS, followed by 1× SSC/0.1% SDS and finally 0.1× SSC/0.1% SDS. Methods for nucleic acid hybridizations are described in Meinkoth and Wahl, 1984 Anal. Biochem. 138:267-284; Current Protocols in Molecular Biology, Chapter 2, Ausubel et al., Eds., Greene Publishing and Wiley-Interscience, New York, 1995; and Tijssen, Laboratory Techniques in Biochemistry and Molecular Biology: Hybridization with Nucleic Acid Probes, Part I, Chapter 2, Elsevier, New York, 1993. Preferably, an isolated nucleic acid molecule of the invention that hybridizes under stringent or highly stringent conditions to a sequence of SEQ ID NO:1, SEQ ID NO:3, SEQ ID NO:5, SEQ ID NO:7 or SEQ ID NO:8 corresponds to a naturally occurring nucleic acid molecule. As used herein, a “naturally occurring” nucleic acid molecule refers to an RNA or DNA molecule having a nucleotide sequence that occurs in nature (e.g., encodes a natural polypeptide).

Using the above-described methods, and others known to those of skill in the art, one of ordinary skill in the art can isolate homologs of the polypeptides comprising an amino acid sequence shown in SEQ ID NO:2, SEQ ID NO:4, SEQ ID NO:6, or SEQ ID NO:9. One subset of these homologs is allelic variants. As used herein, the term “allelic variant” refers to a nucleotide sequence containing polymorphisms that lead to changes in the amino acid sequences of an isopentenyl transferase, a tryptophan monooxygenase or an indole acetamide hydrolase and that exist within a natural population (e.g., a plant species or variety). Such natural allelic variations can typically result in 1-5% variance in an isopentenyl transferase, a tryptophan monooxygenase or an indole acetamide hydrolase nucleic acid. Allelic variants are intended to be within the scope of the invention.

Moreover, nucleic acid molecules encoding isopentenyl transferase, tryptophan monooxygenase and indole acetamide hydrolase polynucleotides from the same or other species such as analogs, orthologs and paralogs, are intended to be within the scope of the present invention. As used herein, the term “analogs” refers to two nucleic acids that have the same or similar function, but that have evolved separately in unrelated organisms. As used herein, the term “orthologs” refers to two nucleic acids from different species, but that have evolved from a common ancestral gene by speciation. Normally, orthologs encode polypeptides having the same or similar functions. As also used herein, the term “paralogs” refers to two nucleic acids that are related by duplication within a genome. Paralogs usually have different functions, but these functions may be related (Tatusov, R. L. et al., 1997 Science 278(5338):631-637).

In addition to naturally-occurring variants of an isopentenyl transferase, a tryptophan monooxygenase or an indole acetamide hydrolase polynueleotide sequence that may exist in the population, the skilled artisan will further appreciate that changes can be introduced by mutation into a nucleotide sequence of SEQ ID NO:1, SEQ ID NO:3, SEQ ID NO:5, SEQ ID NO:7 or SEQ ID NO:8, thereby leading to changes in the amino acid sequence of the encoded isopentenyl transferase, tryptophan monooxygenase or indole acetamide hydrolase polypeptide, without altering the functional activity of the polypeptide. For example, nucleotide substitutions leading to amino acid substitutions at “non-essential” amino acid residues can be made in a sequence of SEQ ID NO:1, SEQ ID NO:3, SEQ ID NO:5, SEQ ID NO:7 or SEQ ID NO:8. A “non-essential” amino acid residue is a residue that can be altered from the wild-type sequence of a polypeptide without altering the activity of said polypeptide, whereas an “essential” amino acid residue is required for polypeptide activity. Other amino acid residues, however, (e.g., those that are not conserved or only semi-conserved in the domain having isopentenyl transferase, tryptophan monooxygenase or indole acetamide hydrolase activity) may not be essential for activity and thus are likely to be amenable to alteration without altering isopentenyl transferase, tryptophan monooxygenase or indole acetamide hydrolase activity.

Accordingly, another aspect of the invention pertains to isopentenyl transferase, tryptophan monooxygenass and indole acetamide hydrolase polypeptides that contain changes in amino acid residues that are not essential for their activity. Such polypeptides differ in amino acid sequence from a sequence contained in SEQ ID NO:2, SEQ ID NO:4, SEQ ID NO:6 or SEQ ID NO:9, yet retain at least one of the isopentenyl transferase, tryptophan monooxygenase or indole acetamide hydrolase activities described herein. An isolated nucleic acid molecule encoding a polypeptide having sequence identity with a polypeptide sequence of SEQ ID NO:2, SEQ ID NO:4, SEQ ID NO:6, or SEQ ID NO:9 can be created by introducing one or more nucleotide substitutions, additions or deletions into a nucleotide sequence of SEQ ID NO:1, SEQ ID NO:3, SEQ ID NO:5, SEQ ID NO:7 or SEQ ID NO:8, such that one or more amino acid substitutions, additions or deletions are introduced into the encoded polypeptide. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Thus, a predicted nonessential amino acid residue in an isopentenyl transferase, tryptophan monooxygenase or indole acetamide hydrolase polypeptide is preferably replaced with another amino acid residue from the same side chain family. Alternatively, in another embodiment, mutations can be introduced randomly along all or part of an isopentenyl transferase, a tryptophan monooxygenase or an indole acetamide hydrolase coding sequence, such as by saturation mutagenesis, and the resultant mutants can be screened for an isopentenyl transferase, tryptophan monooxygenase or indole acetamide hydrolase activity as described herein to identify mutants that retain activity. Following mutagenesis of one of the sequences of SEQ ID NO:1, SEQ ID NO:3, SEQ ID NO:5, SEQ ID NO:7 or SEQ ID NO:8, the encoded polypeptide can be expressed recombinantly and the activity of the polypeptide can be determined by analyzing the plant expressing the polypeptide as described in at least Example 2.

Additionally, optimized isopentenyl transferase, tryptophan monooxygenase and indole acetamide hydrolase polynucleotides can be created. As used herein, “optimized” refers to a nucleic acid that is genetically engineered to increase its expression in a given plant or animal. To provide plant optimized polynucleotides, the DNA sequence of the gene can be modified to 1) comprise codons preferred by highly expressed plant genes; 2) comprise an A+T content in nucleotide base composition to that substantially found in plants; 3) form a plant initiation sequence, 4) to eliminate sequences that cause destabilization, inappropriate polyadenylation, degradation and termination of RNA, or that form secondary structure hairpins or RNA splice sites. Increased expression of polynucleotides in plants can be achieved by utilizing the distribution frequency of codon usage in plants in general or a particular plant. Methods for optimizing nucleic acid expression in plants can be found in EPA 0359472; EPA 0385962; PCT Application No. WO 91/16432; U.S. Pat. No. 5,380,831; U.S. Pat. No. 5,436,391; Perlack et al., 1991 Proc. Natl. Acad. Sci. USA 88:3324-3328; and Murray et al., 1989 Nucleic Acids Res. 17:477-498.

As used herein, “frequency of preferred codon usage” refers to the preference exhibited by a specific host cell in usage of nucleotide codons to specify a given amino acid. To determine the frequency of usage of a particular codon in a gene, the number of occurrences of that codon in the gene is divided by the total number of occurrences of all codons specifying the same amino acid in the gene. Similarly, the frequency of preferred codon usage exhibited by a host cell can be calculated by averaging frequency of preferred codon usage in a large number of genes expressed by the host cell. It is preferable that this analysis be limited to genes that are highly expressed by the host cell. The percent deviation of the frequency of preferred codon usage for a synthetic gene from that employed by a host cell is calculated first by determining the percent deviation of the frequency of usage of a single codon from that of the host cell followed by obtaining the average deviation over all codons. As defined herein, this calculation includes unique codons (i.e., ATG and TGG). In general terms, the overall average deviation of the codon usage of an optimized gene from that of a host cell is calculated using the equation 1A=n=1Z Xₙ−Yₙ Xₙ times 100 Z where Xₙ=frequency of usage for codon n in the host cell; Yₙ=frequency of usage for codon n in the synthetic gene, n represents an individual codon that specifies an amino acid and the total number of codons is Z. The overall deviation of the frequency of codon usage, A, for all amino acids should preferably be less than about 25%, and more preferably less than about 10%.

Hence, a polynucleotide can be optimized such that its distribution frequency of codon usage deviates, preferably, no more than 25% from that of highly expressed plant genes and, more preferably, no more than about 10%. In addition, consideration is given to the percentage G+C content of the degenerate third base (monocotyledons appear to favor G+C in this position, whereas dicotyledons do not). It is also recognized that the XCG (where X is A, T, C, or G) nucleotide is the least preferred codon in dicots whereas the XTA codon is avoided in both monocots and dicots. Optimized polynucleotides of this invention also preferably have CG and TA doublet avoidance indices closely approximating those of the chosen host plant (i.e., a crop plant such as canola). More preferably these indices deviate from that of the host by no more than about 10-15%.

As described above, the invention provides an isolated recombinant expression vector comprising an isopentenyl transferase, a tryptophan monooxygenase or an indole acetamide hydrolase polynucleotides operably linked to a tissue-preferred promoter or a developmental stage-preferred promoter. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990) or see: Gruber and Crosby, in: Methods in Plant Molecular Biology and Biotechnology, eds. Glick and Thompson, Chapter 7, 89-108, CRC Press: Boca Raton, Fla., including the references therein. Regulatory sequences include those that direct constitutive expression of a nucleotide sequence in many types of host cells and those that direct expression of the nucleotide sequence only in certain host cells or under certain conditions. It will be appreciated by those skilled in the art that the design of the expression vector can depend on such factors as the choice of the host cell to be transformed, the level of expression of polypeptide desired, etc. The expression vectors of the invention can be introduced into host cells to thereby produce polypeptides or peptides, including fusion polypeptides or peptides, encoded by nucleic acids as described herein.

Expression of polypeptides in prokaryotes is most often carried out with vectors containing constitutive or inducible promoters directing the expression of either fusion or non-fusion polypeptides. Fusion vectors add a number of amino acids to a polypeptide encoded therein, usually to the amino terminus of the recombinant polypeptide but also to the C-terminus or fused within suitable regions in the polypeptides. Such fusion vectors typically serve three purposes: 1) to increase expression of a recombinant polypeptide; 2) to increase the solubility of a recombinant polypeptide; and 3) to aid in the purification of a recombinant polypeptide by acting as a ligand in affinity purification. Often, in fusion expression vectors, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith, D. B. and Johnson, K. S., 1988 Gene 67:31-40), pMAL (New England Biolabs, Beverly, Mass.) and pRIT5 (Pharmacia, Piscataway, N.J.) which fuse glutathione S-transferase (GST), maltose E binding polypeptide, or polypeptide A, respectively, to the target recombinant polypeptide. 5′ and 3′ untranslated regions and introns that can also be used to modulate expression in specific ways. For example, the pea fed1 5′ UTR and first ⅓^{rd} if the coding region is a light regulated element.

In a preferred embodiment of the present invention, the isopentenyl transferase, tryptophan monooxygenase or indole acetamide hydrolase polynucleotides are expressed in plants and plants cells such as unicellular plant cells (such as algae) (see Falciatore et al., 1999 Marine Biotechnology 1(3):239-251) and plant cells from higher plants (e.g., the spermatophytes, such as crop plants). In a more preferred embodiment, the isopentenyl transferase, tryptophan monooxygenase or indole acetamide hydrolase polynucleotides are expressed in Brassica napus plants or Zea mays plants. Such polynucleotides may be “introduced” into a plant cell by any means, including transfection, transformation or transduction, electroporation, particle bombardment, biolistics, agroinfection and the like.

In one embodiment of the present invention, transfection of an isopentenyl transferase, tryptophan monooxygenase or indole acetamide hydrolase polynucleotide into a plant is achieved by Agrobacterium mediated gene transfer. One transformation method known to those of skill in the art is the dipping of a flowering plant into an Agrobacteria solution, wherein the Agrobacteria contain the expression vector comprising an isopentenyl transferase, a tryptophan monooxygenase or an indole acetamide hydrolase polynucleotide, followed by breeding of the transformed gametes. Other suitable methods for transforming or transfecting host cells including plant cells can be found in Sambrook, et al., (Molecular Cloning: A Laboratory Manual. 2^{nd}, ed., Cold Spring Harbor Laboratory, Cold Spring Harbor Laboratory Press, Cold Spring Harbor, N.Y., 1989) and other laboratory manuals such as Methods in Molecular Biology, 1995, Vol. 44, Agrobacterium protocols, ed: Gartland and Davey, Humana Press, Totowa, N.J. Agrobacterium mediated plant transformation can be performed using for example the GV3101(pMP90) (Koncz and Schell, 1986 Mol. Gen. Genet. 204:383-396) or LBA4404 (Clontech) Agrobacterium tumefaciens strain.

Transformation can be performed by standard transformation and regeneration techniques (Deblaere et al., 1994 Nucl. Acids. Res. 13:4777-4788; Gelvin, Stanton B. and Schilperoort, Robert A, Plant Molecular Biology Manual, 2^{nd} Ed.—Dordrecht: Kluwer Academic Publ., 1995.—in Sect., Ringbuc Zentrale Signatur: BT11-P ISBN 0-7923-2731-4; Glick, Bernard R.; Thompson, John E., Methods in Plant Molecular Biology and Biotechnology, Boca Raton: CRC Press, 1993 360 S., ISBN 0-8493-5164-2). For example, rapeseed can be transformed via cotyledon or hypocotyl transformation (Moloney et al., 1989 Plant cell Report 8:238-242; De Block et al., 1989 Plant Physiol. 91:694-701). Use of antibiotica for Agrobacterium and plant selection depends on the binary vector and the Agrobacterium strain used for transformation. Rapeseed selection is normally performed using kanamycin as selectable plant marker. Agrobacterium mediated gene transfer to flax can be performed using, for example, a technique described by Mlynarova et al., 1994 Plant Cell Report 13:282-285. Additionally, transformation of soybean can be performed using for example a technique described in European Patent No. 0424 047, U.S. Pat. No. 5,322,783, European Patent No. 0397 687, U.S. Pat. No. 5,376,543 or U.S. Pat. No. 5,169,770. Transformation of maize can be achieved by particle bombardment, polyethylene glycol mediated DNA uptake or via the silicon carbide fiber technique. (See, for example, Freeling and Walbot “The maize handbook” Springer Verlag: New York (1993) ISBN 3-540-97826-7). A specific example of maize transformation is found in U.S. Pat. No. 5,990,387 and a specific example of wheat transformation can be found in PCT Application No. WO 93/07256.

According to the present invention, the introduced isopentenyl transferase, tryptophan monooxygenase or indole acetamide hydrolase polynucleotide may be maintained in the plant cell stably if it is incorporated into a non-chromosomal autonomous replicon or integrated into the plant chromosomes. Alternatively, the introduced polynucleotide may be present on an extra-chromosomal non-replicating vector and be transiently expressed or transiently active.

Whether present in an extra-chromosomal non-replicating vector or a vector that is integrated into a chromosome, the isopentenyl transferase, tryptophan monooxygenase or indole acetamide hydrolase polynucleotide preferably resides in a plant expression cassette. A plant expression cassette preferably contains regulatory sequences capable of driving gene expression in plant cells that are operably linked so that each sequence can fulfill its function, for example, termination of transcription by polyadenylation signals. Preferred polyadenylation signals are those originating from Agrobacterium tumefaciens T-DNA such as the gene 3 known as octopine synthase of the Ti-plasmid pTiACH5 (Gielen et al., 1984 EMBO J. 3:835) or functional equivalents thereof but also all other terminators functionally active in plants are suitable. As plant gene expression is very often not limited on transcriptional levels, a plant expression cassette preferably contains other operably linked sequences like translational enhancers such as the overdrive-sequence containing the 5′-untranslated leader sequence from tobacco mosaic virus enhancing the polypeptide per RNA ratio (Gallie et al., 1987 Nucl. Acids Research 15:8693-8711). Examples of plant expression vectors include those detailed in: Becker, D., Kemper, E., Schell, J. and Masterson, R., 1992 New plant binary vectors with selectable markers located proximal to the left border, Plant Mol. Biol. 20:1195-1197; and Bevan, M. W., 1984 Binary Agrobacterium vectors for plant transformation, Nucl. Acid. Res. 12:8711-8721; Vectors for Gene Transfer in Higher Plants; in: Transgenic Plants, Vol. 1, Engineering and Utilization, eds.: Kung and R. Wu, Academic Press, 1993, S. 15-38.

Another aspect of the invention pertains to host cells into which a recombinant expression vector or expression cassette of the invention has been introduced. The terms “host cell” and “recombinant host cell” are used interchangeably herein. It is understood that such terms refer not only to the particular subject cell but they also apply to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell, however, a preferred host cell is a plant cell.

Throughout this application, various publications are referenced. The disclosures of all of these publications and those references cited within those publications in their entireties are hereby incorporated by reference into this application in order to more fully describe the state of the art to which this invention pertains.

It should also be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims.

### EXAMPLES

### Example 1

T-DNA Oncogene Vector Constructs

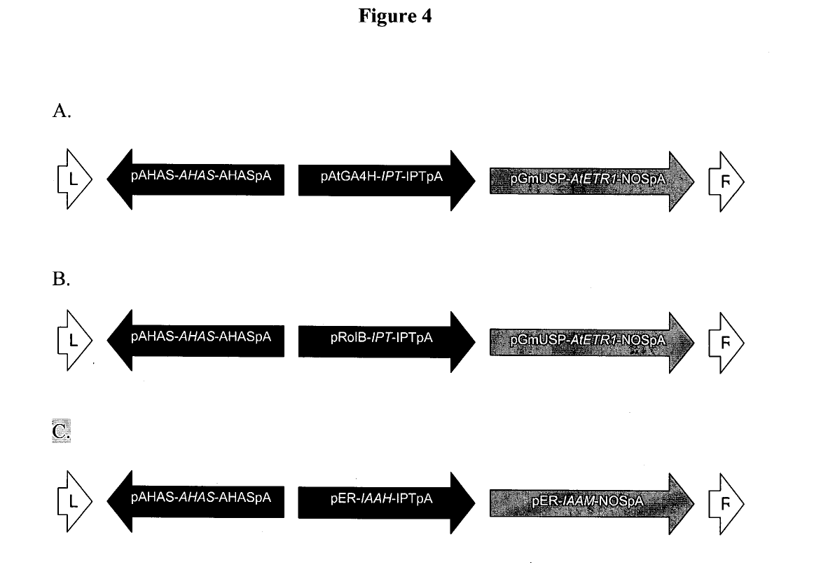

Several specific examples of T-DNA oncogene expression vectors encompassed by the present invention are shown in FIGS. 4(A-B). FIG. 4A shows a construct containing an ipt oncogene from Agrobacterium (IPT) linked to a GA4H promoter from Arabidopsis (pAtGA4H) and an ipt polyadenylation signal. The FIG. 4A construct also contains an AHAS gene with its associated promoter and polyadenylation signal as well as an ethylene response gene from Arabidopsis operably linked to a soybean unknown seed promoter and an Agrobacterium nopaline synthase terminator. This construct is used to achieve increased seedling vigor.

FIG. 4B shows a construct containing an ipt oncogene from Agrobacterium (IPT) linked to a rolB derived promoter from Agrobacterium (pRolB) and an ipt polyadenylation signal. The FIG. 4B construct also contains an AHAS gene with its associated promoter and polyadenylation signal as well as an ethylene response gene from Arabidopsis operably linked to a soybean unknown seed promoter and an Agrobacterium nopaline synthase terminator. This construct is used to achieve increased root mass and/or increased resistance to drought. FIG. 4C shows a construct containing an iaaH oncogene from Agrobacterium (IAAH) operably linked to an A. thaliana ERECTA promoter (pER) and an ipt polyadenylation signal (IPTpA) in tandem with an iaaM oncogene from Agrobacterium (IAAM) operably linked to an A. thaliana ERECTA promoter (pER) and an nos polyadenylation signal (NOSpA).

### Example 2

Transformation of Agrobacteria and Plants With the ipt, iaaH and iaaM T-DNA Oncogenes

Agrobacterium Transformation

Recombinant expression vectors containing the ipt, iaaH and iaaM T-DNA oncogenes are transformed into Agrobacterium tumefaciens strains such as C58C1 and PMP90 according to standard conditions (Hoefgen and Willmitzer, 1990 Plant Science; 66: 221-230). The binary vector plasmids were mobilized into Agrobacterium via electroporation. Electroporations were done according to Mattanovich et al., (1989) and Wen-jun and Forde (1989) with the modifications indicated below. Agrobacterium tumefaciens C58C1 (pMP90) was grown overnight at 28° C. in 5 mL YEB. This overnight culture was used to inoculate 100 mL of YEB medium (Sambrook et al., 1989) and incubated at 29° C. until the optical density at 600 nm reached 0.5-0.7. The culture was then chilled on ice for 10 minutes and centrifuged at 11,827 g (4° C., 10 min.). The pelleted cells were immediately returned to ice after the spent medium was decanted, and washed four times in 10 mL of ice cold sterile 10% glycerol. After washing, the cells were resuspended in 1 mL of ice cold sterile 10% glycerol, aliquoted, and snap frozen in liquid N₂. The resulting electrocompetent cells were stored at −70° C. until use.

Electroporations were preformed as follows. Electrocompetent Agrobacterium cells were thawed on ice. In an ice cold, sterile cuvette (BioRad Gene Pulser; gap distance of 0.2 cm), 40 μL of cells (in 10% glycerol) were mixed with 1-2 μL of binary vector plasmid DNA solution, and electroporated at 2.50 kV, 25 μF, and 600 Ω. One milliliter of ice cold YEB was added immediately after electroporation, and the cells were returned to ice for 2-30 minutes. After a 1 hr. recovery period at 29° C. with gentle shaking (ca. 100 rpm), the transformed Agrobacterium were plated onto YEB medium supplemented with 1.5% agar and 50 mg/L kanamycin. After 2 days, colonies were selected for analysis.

All Agrobacterium cultures were confirmed to contain the appropriate binary vector plasmid by restriction digest analysis of plasmid DNA (Sambrook et al., 1989) minipreparations. A standard E. coli alkaline lysis procedure (Sambrook et al., 1989) was adapted for this purpose by supplementing the resuspension buffer with ca. 20 mg/mL lysozyme (Roche). For transformation protocols, Agrobacterium cultures were grown overnight in LB medium (Sambrook et al., 1989) at 28° C.

Plant Transformation

Arabidopsis thaliana ecotype C24 are grown and transformed according to standard conditions (Bechtold, 1993 Acad. Sci. Paris. 316:1194-1199; Bent et al., 1994 Science 265:1856-1860). Floral dip transformation (FDT) is performed on Arabidopsis thaliana ecotype Columbia (Col0) plants sown in screen covered pots (Bechtold et al., 1993; Bechtold and Pelletier, 1998; Bent et al, 1994). The plants are germinated and grown with an 24 hr., 22° C. day. Cool white fluorescent lamps provided ca. 100 μE/m²s at plant level. An overnight culture of Agrobacterium tumefaciens C58C1 (pMP90; Koncz and Schell, 1986; Hinchee et al., 1988) transformed by electroporation with the pBPS EW051 binary vector plasmid is used to inoculate YEP medium (Sambrook et al., 1989) supplemented with 50 mg/L rifamycin, 50 mg/L gentamycin, and 100 mg/L streptomycin. The Agrobacterium cells are grown first in 2.5 mL overnight at 28° C. with shaking at 275 rpm. The overnight culture is used to inoculate 250 mL YEP culture which is grown overnight at 28° C. with shaking at 275 rpm. The bacteria are then pelleted by centrifugation (30 min., 3500 rpm) and resuspended in 0.25 L of FDM (0.5× MS salts, 5% sucrose, 0.05% Silwet L-77). Plants are dipped when the bolts reach 5-10 cm tall by inverting and submerging the plants and bolts in the Agrobacterium resuspended in FDM and shaded overnight. Finally, the plants are drained and placed in a growth chamber set for 16 hr., 23° C. day and 21° C. night.

Brassica species are transformed using a canola cotyledonary petiole transformation protocol (adapted from Plant Science Sverige AB Protocol) or a Canola Cotyledonary Petiole TAT Protocol which use different methods of Agrobacterium inoculation. Canola seeds are surface sterilized and germinated in vitro on 0.5× MS medium with 1% sucrose and 0.7% Phytagar. Incubate seeds at 23° C. for 5-6 days. Cotyledonary petioles with fully unfolded cotyledons are cut where at their hypocotyl as close as possible to the apical meristem without including it. Inoculated by dipping into a suspension of Agrobacterium diluted with LB medium from an OD₆₀₀ of 1.0-1.5 to an OD₆₀₀ 0.5. Alternatively, inoculate petiole explants by immersing their cut ends for 15 minutes into a suspension of Agrobacterium diluted with liquid MSBAP3A medium (MS salts & vitamins, 3 mg/L BAP, 40 μM acetosyringone) from an OD₆₀₀ of 1.0-1.5 to an OD₆₀₀ 0.5. Plate inoculated explants on solid MSBAP3 medium (MS salts & vitamins, 3 mg/L BAP, 40 μM acetosyringone, 0.7% Phytagar) with the cotyledons suspended above the medium and incubate at the 23° C. After 3 days of co-cultivation at 23° C., explants are transferred to MSBAP3 medium supplemented with 300 mg/L carbenicillin. After 7 days of recovery at 23° C., explants are then transferred to selection on MSBAP3 supplemented with 300 mg/L carbenicillin and a lethal concentration of IMI herbicide (>25 nM Pursuit). Incubate explants at 23° C. and transfer to fresh media every two weeks. After two or more weeks, transfer any green shoots obtained to MS medium with/or without 0.5 mg/L BAP and supplemented with 300 mg/L carbenicillin and selection. Incubate shoot explants at 23° C. and transfer to fresh media every month as long as they continue to regenerate. Once roots develop, the plantlets are transferred to soil.

### Example 3

Screening Plants Containing an ipt T-DNA Oncogene for Increased Resistance to Drought

Ti seeds from plants generated according to Example 2 are sterilized according to standard protocols (Xiong et al., 1999, Plant Molecular Biology Reporter 17:159-170). Seeds are plated on ½ Murashige and Skoog media (MS) pH 5.7 with KOH (Sigma-Aldrich), 0.6% agar and supplemented with 1% sucrose, 150 μg/ml gentamycin (Sigma-Aldrich) and 2 μg/ml benomyl (Sigma-Aldrich). Seeds on plates are vernalized for four days at 4° C. The seeds are germinated in a climatic chamber at an air temperature of 22° C. and light intensity of 40 micromols^{-1m-2} (white light; Philips TL 65W/25 fluorescent tube) and 16 hours light and 8 hours dark day length cycle. Transformed seedlings are selected after 14 days and transferred to ½ MS media pH 5.7 with KOH 0.6% agar plates supplemented with 1% sucrose, 0.5 g/L MES (Sigma-Aldrich), and 2 μg/ml benomyl (Sigma-Aldrich) and allowed to recover for five to seven days.

T1 seedlings are transferred to dry, sterile filter paper in a petri dish and allowed to desiccate for two hours at 80% RH (relative humidity) in a Sanyo Growth Cabinet MLR-350H, micromols^{-1m2} (white light; Philips TL 65W/25 fluorescent tube). The RH is then decreased to 60%, and the seedlings are desiccated further for eight hours. Seedlings are then removed and placed on ½ MS 0.6% agar plates supplemented with 2 μg/ml benomyl (Sigma-Aldrich) and 0.5 g/L MES (Sigma-Aldrich) and scored after five days.

Transgenic plants transformed with the ipt T-DNA oncogenes are then screened for their improved drought tolerance demonstrating that the transgenic expression confers drought tolerance.

### Example 4

Engineering Corn Plants Having Reduced Branching by Expressing the iaaH and iaaM T-DNA Oncogenes Therein

Constructs containing the iaaH and iaaM T-DNA oncogenes as shown in Example 1 above are used to transform corn as described below. Transformation of maize (Zea Mays L.) is performed with the method described by Ishida et al., (1996 Nature Biotech 14745-50). Immature embryos are co-cultivated with Agrobacterium tumefaciens that carry “super binary” vectors, and transgenic plants are recovered through organogenesis. This procedure provides a transformation efficiency of between 2.5% and 20%. The transgenic plants are then screened for their decreased branching.

We claim:

1. A method of improving plant performance, comprising:
a) transforming one or more plant cells with an expression cassette comprising a nucleic acid encoding an isopentenyl transferase, wherein the isopentenyl transferase nucleic acid is operably linked to a developmental stage-preferred promoter or a tissue-preferred promoter and wherein said developmental stage-preferred promoter is not a senescence-preferred promoter; and
b) generating from the one or more plant cells a transgenic plant comprising the expression cassette.

2. The method of claim 1, wherein the nucleic acid encodes an Agrobacterium tumefaciens T-DNA isopentenyl transferase.

3. The method of claim 1, wherein the nucleic acid comprises a polynucleotide sequence selected from the group consisting of:
a) a polynucleotide of SEQ ID NO:1;
b) a polynucleotide encoding a polypeptide of SEQ ID NO:2;
c) a polynucleotide having at least 80% sequence identity with the nucleotide sequence of SEQ ID NO:1;
d) a polynucleotide encoding a polypeptide having at least 80% sequence identity with the polypeptide of SEQ ID NO:2; and
e) a polynucleotide hybridizing under stringent conditions to the nucleotide sequence of SEQ ID NO:1.

4. A method of improving plant performance, comprising:
a) transforming one or more plant cells with one or more nucleic acids encoding a tryptophan monooxygenase and/or an indole acetamide hydrolase, wherein the tryptophan monooxygenase nucleic acid and the indole acetamide hydrolase nucleic acid are operably linked to one or more tissue-preferred promoters and are expressed in the one or more plant cells; and
b) generating from the one or more plant cells a transgenic plant comprising the tryptophan monooxygenase nucleic acid and the indole acetamide hydrolase nucleic acid.

5. The method of claim 4, wherein the nucleic acid encoding a tryptophan monooxygenase comprises a polynucleotide sequence selected from the group consisting of:
a) a polynucleotide of SEQ ID NO:3;
b) a polynucleotide encoding a polypeptide of SEQ ID NO:4;
c) a polynucleotide having at least 80% sequence identity with the nucleotide sequence of SEQ ID NO:3;
d) a polynucleotide encoding a polypeptide having at least 80% sequence identity with the polypeptide of SEQ ID NO:4; and
e) a polynucleotide hybridizing under stringent conditions to the nucleotide sequence of SEQ ID NO:3.

6. The method of claim 4, wherein the nucleic acid encoding an indole acetamide hydrolase nucleic acid encodes an Agrobacterium tumefaciens T-DNA indole acetamide hydrolase.

7. The method of claim 4, wherein the nucleic acid encoding an indole acetamide hydrolase comprises a polynucleotide sequence selected from the group consisting of:
a) a polynucleotide of SEQ ID NO:5;
b) a polynucleotide encoding a polypeptide of SEQ ID NO:6;
c) a polynucleotide having at least 80% sequence identity with the nucleotide sequence of SEQ ID NO:5;
d) a polynucleotide encoding a polypeptide having at least 80% sequence identity with the polypeptide of SEQ ID NO:6; and
e) a polynucleotide hybridizing under stringent conditions to the nucleotide sequence of SEQ ID NO:5.

8. The method of claim 4, wherein the nucleic acid encoding an indole acetamide hydrolase comprises a polynucleotide sequence selected from the group consisting of:
a) a polynucleotide of SEQ ID NO:7 or SEQ ID NO:8;
b) a polynucleotide encoding a polypeptide of SEQ ID NO:9;
c) a polynucleotide having at least 80% sequence identity with the nucleotide sequence of SEQ ID NO:7 or SEQ ID NO:8;
d) a polynucleotide encoding a polypeptide having at least 80% sequence identity with the polypeptide of SEQ ID NO:9; and
e) a polynucleotide hybridizing under stringent conditions to the nucleotide sequence of SEQ ID NO:7 or SEQ ID NO:8.

9. The method of claim 8, wherein the nucleic acid encoding an indole acetamide hydrolase comprises a polynucleotide sequence of SEQ ID NO:7 or SEQ ID NO:8.

10. The method of claim 8, wherein the nucleic acid encoding an indole acetamide hydrolase comprises a polynucleotide sequence encoding a polypeptide of SEQ ID NO:9.

11. The method of claim 8, wherein the nucleic acid encoding an indole acetamide hydrolase comprises a polynucleotide sequence having at least 80% sequence identity with the nucleotide sequence of SEQ ID NO:7 or SEQ ID NO:8.

12. The method of claim 8, wherein the nucleic acid encoding an indole acetamide hydrolase comprises a polynucleotide sequence having at least 80% sequence identity with the polypeptide of SEQ ID NO:9.

13. The method of claim 8, wherein the nucleic acid encoding an indole acetamide hydrolase comprises a polynucleotide sequence hybridizing under stringent conditions to the nucleotide sequence of SEQ ID NO:7 or SEQ ID NO:8.

14. The method of claim 4, wherein the tissue-preferred promoter is a meristem-preferred promoter.

15. An isolated expression cassette comprising a nucleic acid encoding an isopentenyl transferase, Wherein the nucleic acid is operably linked to a developmental stage-preferred promoter or a tissue-preferred promoter.

16. An isolated expression cassette, comprising a nucleic acid encoding a tryptophan monooxygenase, wherein the nucleic acid is operably linked to a tissue-preferred promoter.

17. An isolated expression cassette, comprising a nucleic acid encoding an indole acetamide hydrolase, wherein the nucleic acid is operably linked to a first tissue-preferred promoter.

18. The isolated expression cassette of claim 17, wherein the nucleic acid encodes an Agrobacterium tumefaciens T-DNA indole acetamide hydrolase.

19. The isolated expression cassette of claim 17, wherein the tissue-preferred promoter is a meristem-preferred promoter.

20. The isolated expression cassette of claim 17, further comprising a second nucleic acid encoding a tryptophan monooxygenases, wherein said second nucleic acid is operably linked to the first tissue-preferred promoter.

21. The isolated expression cassette of claim 17, further comprising a second nucleic acid encoding a tryptophan monooxygenases, wherein said second nucleic acid is operably linked to a second tissue-preferred promoter.

22. The isolated expression cassette of claim 17, wherein the nucleic acid comprises a polynucleotide sequence selected from the group consisting of:
a) a polynucleotide of SEQ ID NO:7 or SEQ ID NO:8;
b) a polynucleotide encoding a polypeptide of SEQ ID NO:9;
c) a polynucleotide having at least 80% sequence identity with the nucleotide sequence of SEQ ID NO:7 or SEQ ID NO:8;
d) a polynucleotide encoding a polypeptide having at least 80% sequence identity with the polypeptide of SEQ ID NO:9; and
e) a polynucleotide hybridizing under stringent conditions to the nucleotide sequence of SEQ ID NO:7 or SEQ ID NO:8.

23. The isolated expression cassette of claim 22, wherein the nucleic acid comprises a polynucleotide sequence of SEQ ID NO:7 or SEQ ID NO:8.

24. The isolated expression cassette of claim 22, wherein the nucleic acid comprises a polynucleotide sequence encoding a polypeptide of SEQ ID NO:9.

25. The isolated expression cassette of claim 22, wherein the nucleic acid comprises a polynucleotide sequence having at least 80% sequence identity with the nucleotide sequence of SEQ ID NO:7 or SEQ ID NO:8.

26. The isolated expression cassette of claim 22, wherein the nucleic acid comprises a polynucleotide sequence encoding a polypeptide having at least 80% sequence identity with the polypeptide of SEQ ID NO:9.

27. The isolated expression cassette of claim 22, wherein the nucleic acid comprises a polynucleotide sequence hybridizing under stringent conditions to the nucleotide sequence of SEQ ID NO:7 or SEQ ID NO:8.

28. A transgenic plant cell comprising the isolated expression cassette of claim 22.

29. A transgenic plant comprising the plant cell of claim 28.

30. The transgenic plant of claim 29, wherein the plant is a monocot.

31. The transgenic plant of claim 29, wherein the plant is a dicot.

32. The transgenic plant of claim 29, wherein the plant is selected from the group consisting of maize, wheat, rye, oat, triticale, rice, barley, soybean, peanut, cotton, rapeseed, canola, manihot, pepper, sunflower, tagetes, solanaceous plants, potato, tobacco, eggplant, tomato, Vicia species, pea, alfalfa, coffee, cacao, tea, Salix species, oil palm, coconut, and perennial grass.

33. The transgenic plant of claim 29, wherein the plant is a Brassica napus plant.

34. The transgenic plant of claim 29, wherein the plant is maize.

35. A plant seed produced by the transgenic plant of claim 29, wherein the seed comprises the isolated expression cassette.
